(19) United States
(12) Patent Application Publication
(10) Pub. No.: US 2022/0184117 A1
(43) Pub. Date: Jun. 16, 2022

(54) COMPOSITIONS AND METHODS FOR TREATING, AMELIORATING AND PREVENTING H. PYLORI INFECTIONS

(71) Applicant: Thomas Jujis BORODY, Five Dock (AU)

(72) Inventor: Thomas Julius BORODY, San Diego, CA (US)

(21) Appl. No.: 17/593,046

(22) PCT Filed: Apr. 17, 2020
(86) PCT No.: PCT/AU2020/050384

§ 371 Date: Sep. 7, 2021

Related U.S. Application Data
(60) Provisional application No. 62/835,996, filed on Apr. 18, 2019.

(51) Int. Cl. A61K 33/245; A61K 31/4184; A61K 31/43; A61K 31/7048; A61K 31/506; A61K 31/437

(52) U.S. Cl. CPC A61K 33/245; A61K 31/4184; A61K 31/437; A61K 31/7048; A61K 31/506; A61K 31/43

(57) ABSTRACT

In alternative embodiments, provided are therapeutic combinations, including products of manufacture and kits, and methods and uses thereof, using at least one potassium competitive acid blocker or acid pump blocker, or at least one inhibitor of H+/K+ ATPase, for treating, ameliorating, reversing and/or preventing (acting as a prophylaxis) a Helicobacter pylori (H. pylori) infection in an individual in need thereof.

The present application claims priority to U.S. 62/835,996, the entire disclosure of which is incorporated herein by cross-reference.

### FIELD

This invention generally relates to infectious diseases and medicine. In alternative embodiments, provided are therapeutic combinations, including products of manufacture and kits, and methods, using at least one potassium competitive acid blocker or acid pump blocker, or at least one inhibitor of H+/K+ ATPase, for treating, ameliorating, reversing and/or preventing (acting as a prophylaxis) a Helicobacter pylori (H. pylori) (HP) infection in an individual in need thereof.

### BACKGROUND

The history of Helicobacter pylori (H. pylori) (HP) eradication dates back to 1984 when the first triple therapy was developed in Australia by Thomas Borody at the Centre for Digestive Diseases. It consisted of bismuth, tetracycline, and FLAGYL™. Its long term effects were reported in 1989 and it was dispensed to patients as a separately-prescribed combination written on prescriptions by most physicians until the commercial product called HELIDAC™ was placed on the market in several countries. Since that time numerous triple therapies have been described with the one most utilized being a combination consisting of a Proton Pump Inhibitor (PPI), amoxicillin and clarithromycin. However, there has been a progressive fall in the efficacy of this combination due to H. pylori (HP) developing resistance to clarithromycin and hence, alternate therapies are being sought.

While these therapies have achieved some degree of success, a significant number of treatments still remain failures (see for example, Akazawa Y et al. 2016, Therap Adv Gastroenterol, 9:845) which can result in adverse effects in the responding patients and antibiotic resistance in non-responders. To be clinically effective or useful, H. pylori (HP) eradication efficacy of a therapeutic regimen should reach close to or over 90%—otherwise it is really not sufficiently clinically effective. The existing amoxicillin and clarithromycin PPI protocols were initially able to achieve greater than 85% eradication and became the “standard of care protocols” in many parts of the world. However, the rising resistance to clarithromycin has brought an end to the clinical usefulness of this combination in numerous regions of the world. Some eradication rates have fallen from 90% down to less than 50%.

### SUMMARY OF INVENTION

In some embodiments, forms of the invention may include the following.

1. A method for treating, ameliorating, reversing and/or preventing (acting as a prophylaxis) a Helicobacter pylori (H. pylori) infection in an individual in need thereof, comprising:

administering to the individual in need thereof a drug combination, comprising:

at least one potassium-competitive acid blocker (PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

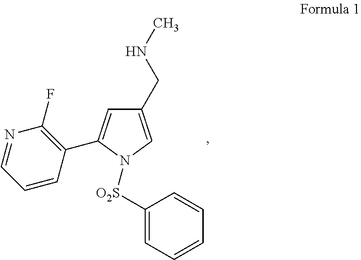 [SMILES: CNCc1cc(-c2cccnc2F)n(S(=O)(=O)c2ccccc2)c1]

a compound having the structure of Formula 2, or equivalent,

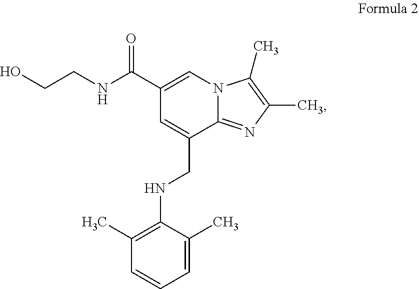 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent.

2. The method of form 1, further comprising co-administering with the potassium competitive acid blocker or the acid pump blocker, or the inhibitor of H+/K+ ATPase, an antimicrobial or antibiotic composition comprising or consisting of:

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally administered to the individual in need thereof at a dose of between about 10 mg to about 450 mg bid or tid;

(c) a clarithromycin, optionally BIAXIN™,
optionally administered to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally administered to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl) -1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally administered to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally administered to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally administered to the individual in need thereof at a daily dose of between about 50 mg to 3 grams;

(h) a levofloxacin, optionally LEVAQUIN™, TAVANIC™, IQUIX™,
optionally administered to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally administered to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally administered to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally administered to the individual in need thereof at a dose of between about 15 mg to about 50 mg per day, or at about 25 to 5000 mg per day;

(l) a furazolidone, optionally FUROXONE™, DEPENDAL-M™,
optionally administered to the individual in need thereof at a dose of between about 5 to about 6000 mg/d;

(m) a rifabutin, optionally MYCOBUTIN™,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally administered to the individual in need thereof at a dose of between about 50 to 2000 mg/day or between about 50 to 2000 mg bid;

(o) a furazolidine, optionally FUROXONE™, DEPENDAL-M™,
optionally administered to the individual in need thereof at a dose of between about 5 mg to about 6000 mg per day;

(p) a bismuth, bismuth salicylate or bismuth subsalicylate, optionally PEPTO-BISMOL™,
optionally administered to the individual in need thereof at a dose of between about 100 mg to about 4000 mg per day;

(q) tetracycline, optionally SUMYCIN™, TETRACYN™, PANMYCIN™,
optionally administered to the individual in need thereof at a dose of between about 60 mg to 5000 mg per day;

(r) a doxycycline, optionally DORYZ™, DOXYHEXA™L, DOXYLIN™,
optionally administered to the individual in need thereof at a dose of between about 60 mg to 5000 mg per day;

(s) a minocycline, optionally MINOCIN™, MINOCYCIN™, AKAMIN™,
optionally administered to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is administered at a dose of between about 500 mg to 5 gm per day, or 250 mg to 6 gm per day.

3. The method of form 1 or form 2, wherein the drug combination is or comprises:

(1) tegoprazan, revaprazan, linaprazan or soraprazan; and, amoxicillin or amoxicillin/clavulanic acid;

(2) tegoprazan, revaprazan, linaprazan or soraprazan; and, clarithromycin;

(3) tegoprazan, revaprazan, linaprazan or soraprazan; clarithromycin; and, amoxicillin or amoxicillin/clavulanic acid;

(4) tegoprazan, revaprazan, linaprazan or soraprazan; and, rifabutin;

(5) tegoprazan, revaprazan, linaprazan or soraprazan; clarithromycin; and, rifabutin;

(6) tegoprazan, revaprazan, linaprazan or soraprazan; amoxicillin or amoxicillin/clavulanic acid; and, rifabutin;

(7) tegoprazan, revaprazan, linaprazan or soraprazan; amoxicillin or amoxicillin/clavulanic acid; clarithromycin; and, rifabutin;

(8) tegoprazan, revaprazan, linaprazan or soraprazan; and, levofloxacin;

(9) tegoprazan, revaprazan, linaprazan or soraprazan; clarithromycin; and, levofloxacin;

(10) tegoprazan, revaprazan, linaprazan or soraprazan; amoxicillin or amoxicillin/clavulanic acid; and, levofloxacin;

(12) tegoprazan, revaprazan, linaprazan or soraprazan; amoxicillin or amoxicillin/clavulanic acid; clarithromycin; and, levofloxacin;

(13) tegoprazan, revaprazan, linaprazan or soraprazan; amoxicillin or amoxicillin/clavulanic acid; rifabutin; and, levofloxacin;

(14) tegoprazan, revaprazan, linaprazan or soraprazan; amoxicillin or amoxicillin/clavulanic acid; rifabutin; clarithromycin; and, levofloxacin;

(15) tegoprazan, revaprazan, linaprazan or soraprazan; amoxicillin or amoxicillin/clavulanic acid; rifabutin; and, tetracycline;

(16) tegoprazan, revaprazan, linaprazan or soraprazan; amoxicillin or amoxicillin/clavulanic acid; rifabutin; clarithromycin; and, tetracycline;

(17) tegoprazan, revaprazan, linaprazan or soraprazan; and, tetracycline;

(18) tegoprazan, revaprazan, linaprazan or soraprazan; clarithromycin; and, tetracycline;

(19) tegoprazan, revaprazan, linaprazan or soraprazan; and, metronidazole or tinidazole;

(20) tegoprazan, revaprazan, linaprazan or soraprazan; clarithromycin; and, metronidazole or tinidazole;

(21) tegoprazan, revaprazan, linaprazan or soraprazan; and, a bismuth, bismuth salicylate or bismuth subsalicylate;

(22) tegoprazan, revaprazan, linaprazan or soraprazan; clarithromycin; and, a bismuth, bismuth salicylate or bismuth subsalicylate;

(23) tegoprazan, revaprazan, linaprazan or soraprazan; tetracycline; and, a bismuth, bismuth salicylate or bismuth subsalicylate;

(24) tegoprazan, revaprazan, linaprazan or soraprazan; tetracycline; clarithromycin; and, a bismuth, bismuth salicylate or bismuth subsalicylate;

(25) tegoprazan, revaprazan, linaprazan or soraprazan; tetracycline; a bismuth, bismuth salicylate or bismuth subsalicylate; and, metronidazole or tinidazole;

(26) tegoprazan, revaprazan, linaprazan or soraprazan; tetracycline; a bismuth, bismuth salicylate or bismuth subsalicylate; clarithromycin; and, metronidazole or tinidazole;

(27) tegoprazan, revaprazan, linaprazan or soraprazan; tetracycline; and, metronidazole or tinidazole;

(28) tegoprazan, revaprazan, linaprazan or soraprazan; tetracycline; clarithromycin; and, metronidazole or tinidazole;

(29) tegoprazan, revaprazan, linaprazan or soraprazan; and, ampicillin;

(30) tegoprazan, revaprazan, linaprazan or soraprazan; clarithromycin; and, ampicillin;

(31) tegoprazan, revaprazan, linaprazan or soraprazan; tetracycline; metronidazole or tinidazole; and, ampicillin;

(32) tegoprazan, revaprazan, linaprazan or soraprazan; tetracycline; metronidazole or tinidazole; clarithromycin; and, ampicillin;

(33) tegoprazan, revaprazan, linaprazan or soraprazan; tetracycline; and, ampicillin;

(34) tegoprazan, revaprazan, linaprazan or soraprazan; tetracycline; clarithromycin; and, ampicillin;

(35) tegoprazan, revaprazan, linaprazan or soraprazan; and, rifabutin; tegoprazan, revaprazan, linaprazan or soraprazan; clarithromycin; and, rifabutin;

(36) tegoprazan, revaprazan, linaprazan or soraprazan; and, rifampicin;

(37) tegoprazan, revaprazan, linaprazan or soraprazan; clarithromycin; and, rifampicin;

(38) tegoprazan, revaprazan, linaprazan or soraprazan; rifabutin; and, rifampicin;

(39) tegoprazan, revaprazan, linaprazan or soraprazan; rifabutin; clarithromycin; and, rifampicin;

(40) tegoprazan, revaprazan, linaprazan or soraprazan; rifabutin; tetracycline, and, rifampicin;

(41) tegoprazan, revaprazan, linaprazan or soraprazan; rifabutin; tetracycline, clarithromycin; and, rifampicin;

(42) tegoprazan, revaprazan, linaprazan or soraprazan; and, clarithromycin; tegoprazan, revaprazan, and, clarithromycin;

(43) tegoprazan, revaprazan, linaprazan or soraprazan; rifabutin; and, clarithromycin;

(44) tegoprazan, revaprazan, linaprazan or soraprazan; and, clarithromycin;

(45) tegoprazan, revaprazan, linaprazan or soraprazan; rifampicin; and, clarithromycin;

(46) rifampicin; and, clarithromycin;

(47) tegoprazan, revaprazan, linaprazan or soraprazan; and, azithromycin;

(48) tegoprazan, revaprazan, linaprazan or soraprazan; clarithromycin; and, azithromycin;

(49) tegoprazan, revaprazan, linaprazan or soraprazan; rifampicin; and, azithromycin;

(50) tegoprazan, revaprazan, linaprazan or soraprazan; clarithromycin; rifampicin; and, azithromycin;

(51) tegoprazan, revaprazan, linaprazan or soraprazan; rifabutin; and, azithromycin;

(52) tegoprazan, revaprazan, linaprazan or soraprazan; rifabutin; clarithromycin; and, azithromycin;

(53) tegoprazan, revaprazan, linaprazan or soraprazan; rifabutin; and, a vonoprazan or a vonoprazan fumarate;

(54) tegoprazan, revaprazan, linaprazan or soraprazan; rifabutin; clarithromycin; and, a vonoprazan or a vonoprazan fumarate;

(55) tegoprazan, revaprazan, linaprazan or soraprazan; rifabutin; and, metronidazole;

(55) tegoprazan, revaprazan, linaprazan or soraprazan; rifabutin; clarithromycin; and, metronidazole;

(56) tegoprazan, revaprazan, linaprazan or soraprazan; rifabutin; and, tinidazole;

(57) tegoprazan, revaprazan, linaprazan or soraprazan; rifabutin; clarithromycin; and, tinidazole;

(58) tegoprazan, revaprazan, linaprazan or soraprazan; rifabutin; and, nitazoxanide;

(59) tegoprazan, revaprazan, linaprazan or soraprazan; rifabutin; clarithromycin; and, nitazoxanide;

(60) tegoprazan, revaprazan, linaprazan or soraprazan; rifabutin; and, furazolidine;

(61) tegoprazan, revaprazan, linaprazan or soraprazan; rifabutin; clarithromycin; and, furazolidine;

(62) tegoprazan, revaprazan, linaprazan or soraprazan; and, ciprofloxacin;

(63) tegoprazan, revaprazan, linaprazan or soraprazan; clarithromycin; and, ciprofloxacin;

(64) tegoprazan, revaprazan, linaprazan or soraprazan; rifabutin; and, ciprofloxacin;

(65) tegoprazan, revaprazan, linaprazan or soraprazan; rifabutin; clarithromycin; and, ciprofloxacin

(66) tegoprazan, revaprazan, linaprazan or soraprazan; and, doxycycline or minocycline; or

(67) tegoprazan, revaprazan, linaprazan or soraprazan; clarithromycin; and, doxycycline or minocycline.

4. The method of any one of the preceding forms, wherein the drug combination comprises a potassium competitive acid blocker and amoxicillin.
5. The method of form 4, wherein the amoxicillin is administered in an amount of at least 3 g/day.
6. The method of any one of the preceding forms, wherein the drug combination comprises a potassium competitive acid blocker, amoxicillin, and clarithromycin.
7. The method of any one of the preceding forms, wherein the drug combination comprises a potassium competitive acid blocker, amoxicillin, and a rifamycin derivative.
8. The method of form 7, wherein the rifamycin derivative is rifabutin.
9. The method of any one of forms 4 to 8, wherein the potassium competitive acid blocker is selected from the group consisting of tegoprazan, revaprazan, and linaprazan.
10. The method of any one of the preceding forms, wherein the drug combination is:

(i) tegoprazan, amoxicillin, and clarithromycin;

(ii) tegoprazan and amoxicillin; or

(iii) revaprazan, amoxicillin, and rifabutin.

11. The method of any one of forms 1 to 3, wherein the drug combination comprises a potassium competitive acid blocker, a tetracycline antibiotic, a nitroimidazole antibiotic, and a bismuth salt.
12. The method of form 11, wherein the drug combination comprises linaprazan, tetracycline, tinidazole, and bismuth subsalicylate.
13. The method of any one of the preceding forms, wherein the drug combination is administered to the individual in need thereof for about 2, 3, 4, 5, 6, 7, 8, 9, 10, 11, 12, 13 or 14 or more days.
14. The method of any one of forms 2 to 13, wherein the antimicrobial or antibiotic composition is administered together with, before and/or after administration of the potassium competitive acid blocker, the acid pump blocker or the inhibitor of H+/K+ ATPase.
15. The method of any one of forms 2 to 14, wherein the antimicrobial or antibiotic composition is formulated together with, or separately from, the potassium competitive acid blocker, the acid pump blocker or the inhibitor of H+/K+ ATPase.
16. The method of any one of the preceding forms, wherein the drug combination is contained in or comprises one or more of a formulation, a pharmaceutical preparation or a pharmaceutical composition.
17. The method of any one of the preceding forms, wherein any one or several of the potassium competitive acid blocker or the acid pump blocker, or the inhibitor of H+/K+ ATPase, or the antimicrobial or antibiotic composition is administered to the individual in need thereof:

(a) at a unit dosage of between about 5 mg to about 5000 mg per day, or

(b) in a unit dosage form of between about 10 mg and 200 mg, or between about between about 40 mg and 100 mg, or between about between about 100 mg and 500 mg, or between about between about 500 mg and 1000 mg, or is about 10, 20, 30, 40, 50, 60, 70, 75, 80, 90 or 100 mg per unit dose,

which optionally can be administered once a day, bid or tid, or a four times a day, five times a day or six times a day or more, regimen.

18. The method of any one of the preceding forms, wherein drug combination is formulated as a chewable delivery vehicle, a gum, a gummy, a candy, a lozenge, an ice cream or an ice, or a yogurt.
19. The method of any one of the preceding forms, wherein drug combination further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
20. The method of any one of the preceding forms, wherein the drug combination further comprises a preservative, a benzoic acid or a potassium sorbate.
21. The method of any one of the preceding forms, wherein the drug combination further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep. fecalis and equivalents.
22. The method of any one of the preceding forms, wherein drug the combination further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
23. The method of any one of the preceding forms, wherein the drug combination further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
24. The method of any one of the preceding forms, wherein the drug combination further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™), atorvastatin (optionally LIPITOR™), pravastatin (optionally PRAVACHOL™), fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
25. The method of any one of the preceding forms, wherein the drug combination is formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation.
26. The method of any one of the preceding forms, wherein the drug combination is contained in a delivery vehicle, product of manufacture, container, syringe, device or bag.
27. The method of any one of the preceding forms, wherein the drug combination is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.
28. A drug combination comprising:

(a) the drug combination as defined in the method of any one of the preceding forms; or

(b)

[symbol]

(i) a drug combination comprising at least one potassium-competitive acid blocker (PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy] -N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

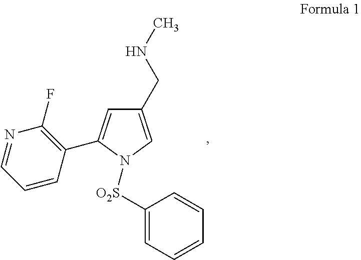 [SMILES: CNCc1cc(-c2cccnc2F)n(S(=O)(=O)c2ccccc2)c1]

a compound having the structure of Formula 2, or equivalent,

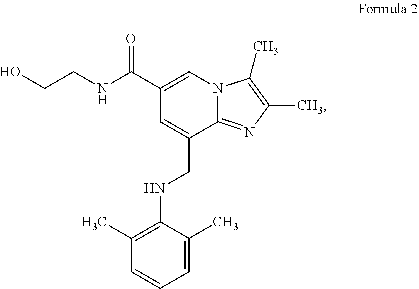 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent; and

(ii)

[symbol]

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally administered to the individual in need thereof at a dose of between about 10 mg to about 450 mg bid or tid;

(c) a clarithromycin, optionally BIAXIN™,
optionally administered to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally administered to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol -3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally administered to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally administered to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally administered to the individual in need thereof at a daily dose of between about 50 mg to 3 grams;

(h) a levofloxacin, optionally LEVAQUIN™, TAVANIC™, IQUIX™,
optionally administered to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally administered to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally administered to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally administered to the individual in need thereof at a dose of between about 15 mg to about 50 mg per day, or at about 25 to 5000 mg per day;

(l) a furazolidone, optionally FUROXONE™, DEPENDAL-M™,
optionally administered to the individual in need thereof at a dose of between about 5 to about 6000 mg/d;

(m) a rifabutin, optionally MYCOBUTIN™,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally administered to the individual in need thereof at a dose of between about 50 to 2000 mg/day or between about 50 to 2000 mg bid;

(o) a furazolidine, optionally FUROXONE™, DEPENDAL-M™,
optionally administered to the individual in need thereof at a dose of between about 5 mg to about 6000 mg per day;

(p) a bismuth, bismuth salicylate or bismuth subsalicylate, optionally PEPTO-BISMOL™,
optionally administered to the individual in need thereof at a dose of between about 100 mg to about 4000 mg per day;

(q) tetracycline, optionally SUMYCIN™, TETRACYN™, PANMYCIN™,
optionally administered to the individual in need thereof at a dose of between about 60 mg to 5000 mg per day;

(r) a doxycycline, optionally DORYZ™, DOXYHEXA™L, DOXYLIN™,
optionally administered to the individual in need thereof at a dose of between about 60 mg to 5000 mg per day;

(s) a minocycline, optionally MINOCIN™, MINOCYCIN™, AKAMIN™,
optionally administered to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is administered at a dose of between about 500 mg to 5 gm per day, or 250 mg to 6 gm per day.

29. A kit or product of manufacture comprising a drug combination as defined in any one of the preceding forms.
30. Use of a drug combination as defined in any one of the preceding forms, in the manufacture of a medicament for treating, ameliorating, reversing and/or preventing (acting as a prophylaxis) a Helicobacter pylori (H. pylori) infection in an individual in need thereof.
31. A drug combination as defined in any one of the preceding forms for use in treating, ameliorating, reversing and/or preventing (acting as a prophylaxis) a Helicobacter pylori (H. pylori) infection in an individual in need thereof.

In some embodiments, forms of the invention may include the following.

1. A drug combination, comprising:

at least one potassium-competitive acid blocker (PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

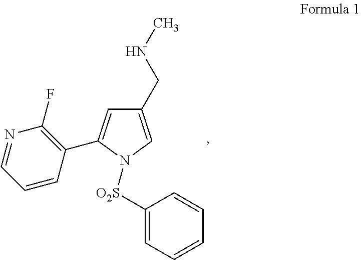 [SMILES: CNCc1cc(-c2cccnc2F)n(S(=O)(=O)c2ccccc2)c1]

a compound having the structure of Formula 2, or equivalent,

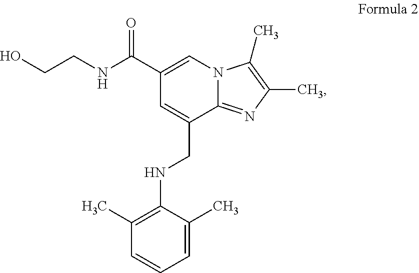 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent,
   for use in treating, ameliorating, reversing and/or preventing (acting as a prophylaxis) a Helicobacter pylori (H. pylori) infection in an individual in need thereof.
   2. The drug combination of form 1, further comprising an antimicrobial or antibiotic composition comprising or consisting of:

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is formulated for administration to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally formulated for administration to the individual in need thereof at a dose of between about 10 mg to about 450 mg bid or tid;

(c) a clarithromycin, optionally BIAXIN™,
optionally formulated for administration to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally formulated for administration to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally formulated for administration to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally formulated for administration to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally formulated for administration to the individual in need thereof at a daily dose of between about 50 mg to 3 grams;

(h) a levofloxacin, optionally LEVAQUIN™, TAVANIC™, IQUIX™,
optionally formulated for administration to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally formulated for administration to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally formulated for administration to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally formulated for administration to the individual in need thereof at a dose of between about 15 mg to about 50 mg per day, or at about 25 to 5000 mg per day;

(l) a furazolidone, optionally FUROXONE™, DEPENDAL-M™,
optionally formulated for administration to the individual in need thereof at a dose of between about 5 to about 6000 mg/d;

(m) a rifabutin, optionally MYCOBUTIN™,
optionally formulated for administration to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally formulated for administration to the individual in need thereof at a dose of between about 50 to 2000 mg/day or between about 50 to 2000 mg bid;

(o) a furazolidine, optionally FUROXONE™, DEPENDAL-M™,
optionally formulated for administration to the individual in need thereof at a dose of between about 5 mg to about 6000 mg per day;

(p) a bismuth, bismuth salicylate or bismuth subsalicylate, optionally PEPTO-BISMOL™,
optionally formulated for administration to the individual in need thereof at a dose of between about 100 mg to about 4000 mg per day;

(q) tetracycline, optionally SUMYCIN™, TETRACYN™, PANMYCIN™,
optionally formulated for administration to the individual in need thereof at a dose of between about 60 mg to 5000 mg per day;

(r) a doxycycline, optionally DORYZ™, DOXYHEXA™L, DOXYLIN™,
optionally formulated for administration to the individual in need thereof at a dose of between about 60 mg to 5000 mg per day;

(s) a minocycline, optionally MINOCIN™, MINOCYCIN™, AKAMIN™,
optionally formulated for administration to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is formulated for administration at a dose of between about 500 mg to 5 gm per day, or 250 mg to 6 gm per day.

3. The drug combination of form 1 or form 2, comprising or consisting of:

(1) tegoprazan, revaprazan, linaprazan or soraprazan; and, amoxicillin or amoxicillin/clavulanic acid;

(2) tegoprazan, revaprazan, linaprazan or soraprazan; and, clarithromycin;

(3) tegoprazan, revaprazan, linaprazan or soraprazan; clarithromycin; and, amoxicillin or amoxicillin/clavulanic acid;

(4) tegoprazan, revaprazan, linaprazan or soraprazan; and, rifabutin;

(5) tegoprazan, revaprazan, linaprazan or soraprazan; clarithromycin; and, rifabutin;

(6) tegoprazan, revaprazan, linaprazan or soraprazan; amoxicillin or amoxicillin/clavulanic acid; and, rifabutin;

(7) tegoprazan, revaprazan, linaprazan or soraprazan; amoxicillin or amoxicillin/clavulanic acid; clarithromycin; and, rifabutin;

(8) tegoprazan, revaprazan, linaprazan or soraprazan; and, levofloxacin;

(9) tegoprazan, revaprazan, linaprazan or soraprazan; clarithromycin; and, levofloxacin;

(10) tegoprazan, revaprazan, linaprazan or soraprazan; amoxicillin or amoxicillin/clavulanic acid; and, levofloxacin;

(12) tegoprazan, revaprazan, linaprazan or soraprazan; amoxicillin or amoxicillin/clavulanic acid; clarithromycin; and, levofloxacin;

(13) tegoprazan, revaprazan, linaprazan or soraprazan; amoxicillin or amoxicillin/clavulanic acid; rifabutin; and, levofloxacin;

(14) tegoprazan, revaprazan, linaprazan or soraprazan; amoxicillin or amoxicillin/clavulanic acid; rifabutin; clarithromycin; and, levofloxacin;

(15) tegoprazan, revaprazan, linaprazan or soraprazan; amoxicillin or amoxicillin/clavulanic acid; rifabutin; and, tetracycline;

(16) tegoprazan, revaprazan, linaprazan or soraprazan; amoxicillin or amoxicillin/clavulanic acid; rifabutin; clarithromycin; and, tetracycline;

(17) tegoprazan, revaprazan, linaprazan or soraprazan; and, tetracycline;

(18) tegoprazan, revaprazan, linaprazan or soraprazan; clarithromycin; and, tetracycline;

(19) tegoprazan, revaprazan, linaprazan or soraprazan; and, metronidazole or tinidazole;

(20) tegoprazan, revaprazan, linaprazan or soraprazan; clarithromycin; and, metronidazole or tinidazole;

(21) tegoprazan, revaprazan, linaprazan or soraprazan; and, a bismuth, bismuth salicylate or bismuth subsalicylate;

(22) tegoprazan, revaprazan, linaprazan or soraprazan; clarithromycin; and, a bismuth, bismuth salicylate or bismuth subsalicylate;

(23) tegoprazan, revaprazan, linaprazan or soraprazan; tetracycline; and, a bismuth, bismuth salicylate or bismuth subsalicylate;

(24) tegoprazan, revaprazan, linaprazan or soraprazan; tetracycline; clarithromycin; and, a bismuth, bismuth salicylate or bismuth subsalicylate;

(25) tegoprazan, revaprazan, linaprazan or soraprazan; tetracycline; a bismuth, bismuth salicylate or bismuth subsalicylate; and, metronidazole or tinidazole;

(26) tegoprazan, revaprazan, linaprazan or soraprazan; tetracycline; a bismuth, bismuth salicylate or bismuth subsalicylate; clarithromycin; and, metronidazole or tinidazole;

(27) tegoprazan, revaprazan, linaprazan or soraprazan; tetracycline; and, metronidazole or tinidazole;

(28) tegoprazan, revaprazan, linaprazan or soraprazan; tetracycline; clarithromycin; and, metronidazole or tinidazole;

(29) tegoprazan, revaprazan, linaprazan or soraprazan; and, ampicillin;

(30) tegoprazan, revaprazan, linaprazan or soraprazan; clarithromycin; and, ampicillin;

(31) tegoprazan, revaprazan, linaprazan or soraprazan; tetracycline; metronidazole or tinidazole; and, ampicillin;

(32) tegoprazan, revaprazan, linaprazan or soraprazan; tetracycline; metronidazole or tinidazole; clarithromycin; and, ampicillin;

(33) tegoprazan, revaprazan, linaprazan or soraprazan; tetracycline; and, ampicillin;

(34) tegoprazan, revaprazan, linaprazan or soraprazan; tetracycline; clarithromycin; and, ampicillin;

(35) tegoprazan, revaprazan, linaprazan or soraprazan; and, rifabutin; tegoprazan, revaprazan, linaprazan or soraprazan; clarithromycin; and, rifabutin;

(36) tegoprazan, revaprazan, linaprazan or soraprazan; and, rifampicin;

(37) tegoprazan, revaprazan, linaprazan or soraprazan; clarithromycin; and, rifampicin;

(38) tegoprazan, revaprazan, linaprazan or soraprazan; rifabutin; and, rifampicin;

(39) tegoprazan, revaprazan, linaprazan or soraprazan; rifabutin; clarithromycin; and, rifampicin;

(40) tegoprazan, revaprazan, linaprazan or soraprazan; rifabutin; tetracycline, and, rifampicin;

(41) tegoprazan, revaprazan, linaprazan or soraprazan; rifabutin; tetracycline, clarithromycin; and, rifampicin;

(42) tegoprazan, revaprazan, linaprazan or soraprazan; and, clarithromycin; tegoprazan, revaprazan, and, clarithromycin;

(43) tegoprazan, revaprazan, linaprazan or soraprazan; rifabutin; and, clarithromycin;

(44) tegoprazan, revaprazan, linaprazan or soraprazan; and, clarithromycin;

(45) tegoprazan, revaprazan, linaprazan or soraprazan; rifampicin; and, clarithromycin;

(46) rifampicin; and, clarithromycin;

(47) tegoprazan, revaprazan, linaprazan or soraprazan; and, azithromycin;

(48) tegoprazan, revaprazan, linaprazan or soraprazan; clarithromycin; and, azithromycin;

(49) tegoprazan, revaprazan, linaprazan or soraprazan; rifampicin; and, azithromycin;

(50) tegoprazan, revaprazan, linaprazan or soraprazan; clarithromycin; rifampicin; and, azithromycin;

(51) tegoprazan, revaprazan, linaprazan or soraprazan; rifabutin; and, azithromycin;

(52) tegoprazan, revaprazan, linaprazan or soraprazan; rifabutin; clarithromycin; and, azithromycin;

(53) tegoprazan, revaprazan, linaprazan or soraprazan; rifabutin; and, a vonoprazan or a vonoprazan fumarate;

(54) tegoprazan, revaprazan, linaprazan or soraprazan; rifabutin; clarithromycin; and, a vonoprazan or a vonoprazan fumarate;

(55) tegoprazan, revaprazan, linaprazan or soraprazan; rifabutin; and, metronidazole;

(55) tegoprazan, revaprazan, linaprazan or soraprazan; rifabutin; clarithromycin; and, metronidazole;

(56) tegoprazan, revaprazan, linaprazan or soraprazan; rifabutin; and, tinidazole;

(57) tegoprazan, revaprazan, linaprazan or soraprazan; rifabutin; clarithromycin; and, tinidazole;

(58) tegoprazan, revaprazan, linaprazan or soraprazan; rifabutin; and, nitazoxanide;

(59) tegoprazan, revaprazan, linaprazan or soraprazan; rifabutin; clarithromycin; and, nitazoxanide;

(60) tegoprazan, revaprazan, linaprazan or soraprazan; rifabutin; and, furazolidine;

(61) tegoprazan, revaprazan, linaprazan or soraprazan; rifabutin; clarithromycin; and, furazolidine;

(62) tegoprazan, revaprazan, linaprazan or soraprazan; and, ciprofloxacin;

(63) tegoprazan, revaprazan, linaprazan or soraprazan; clarithromycin; and, ciprofloxacin;

(64) tegoprazan, revaprazan, linaprazan or soraprazan; rifabutin; and, ciprofloxacin;

(65) tegoprazan, revaprazan, linaprazan or soraprazan; rifabutin; clarithromycin; and, ciprofloxacin

(66) tegoprazan, revaprazan, linaprazan or soraprazan; and, doxycycline or minocycline; or

(67) tegoprazan, revaprazan, linaprazan or soraprazan; clarithromycin; and, doxycycline or minocycline.

4. The drug combination of any one of the preceding forms comprising a potassium competitive acid blocker and amoxicillin.
5. The drug combination of form 4, wherein the amoxicillin is administered in an amount of at least 3 g/day.
6. The drug combination of any one of the preceding forms, comprising a potassium competitive acid blocker, amoxicillin, and clarithromycin.
7. The drug combination of any one of the preceding forms, comprising a potassium competitive acid blocker, amoxicillin, and a rifamycin derivative.
8. The drug combination of form 7, wherein the rifamycin derivative is rifabutin.
9. The drug combination of any one of forms 4 to 8, wherein the potassium competitive acid blocker is selected from the group consisting of tegoprazan, revaprazan, and linaprazan.
10. The drug combination of any one of the preceding forms, comprising:

(i) tegoprazan, amoxicillin, and clarithromycin;

(ii) tegoprazan and amoxicillin; or

(iii) revaprazan, amoxicillin, and rifabutin.

11. The drug combination of any one of forms 1 to 3, comprising a potassium competitive acid blocker, a tetracycline antibiotic, a nitroimidazole antibiotic, and a bismuth salt.
12. The drug combination of form 11, comprising linaprazan, tetracycline, tinidazole, and bismuth subsalicylate.
13. The drug combination of any one of the preceding forms, formulated for administration to the individual in need thereof for about 2, 3, 4, 5, 6, 7, 8, 9, 10, 11, 12, 13 or 14 or more days.
14. The drug combination of any one of forms 2 to 13, wherein the antimicrobial or antibiotic composition is formulated for administration together with, before and/or after the potassium competitive acid blocker, the acid pump blocker or the inhibitor of H+/K+ ATPase.
15. The drug combination of any one of forms 2 to 14, wherein the antimicrobial or antibiotic composition is formulated together with, or separately from, the potassium competitive acid blocker, the acid pump blocker or the inhibitor of H+/K+ ATPase.
16. The drug combination of any one of the preceding forms, wherein the drug combination is contained in or comprises one or more of a formulation, a pharmaceutical preparation or a pharmaceutical composition.
17. The drug combination of any one of the preceding forms, wherein any one or several of the potassium competitive acid blocker or the acid pump blocker, or the inhibitor of H+/K+ ATPase, or the antimicrobial or antibiotic composition is formulated for administration to the individual in need thereof:

(a) at a unit dosage of between about 5 mg to about 5000 mg per day, or

(b) in a unit dosage form of between about 10 mg and 200 mg, or between about between about 40 mg and 100 mg, or between about between about 100 mg and 500 mg, or between about between about 500 mg and 1000 mg, or is about 10, 20, 30, 40, 50, 60, 70, 75, 80, 90 or 100 mg per unit dose,

which optionally is formulated for administration in a once a day, bid or tid, or a four times a day, five times a day or six times a day or more, regimen.

18. The drug combination of any one of the preceding forms, formulated as a chewable delivery vehicle, a gum, a gummy, a candy, a lozenge, an ice cream or an ice, or a yogurt.
19. The drug combination of any one of the preceding forms, further comprising a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
20. The drug combination of any one of the preceding forms, further comprising a preservative, a benzoic acid or a potassium sorbate.
21. The drug combination of any one of the preceding forms, further comprising at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep. fecalis and equivalents.
22. The drug combination of any one of the preceding forms, further comprising at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch.
23. The drug combination of any one of the preceding forms, further comprising an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
24. The drug combination of any one of the preceding forms, further comprising at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™) atorvastatin (optionally LIPITOR™), pravastatin (optionally PRAVACHOL™), fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
25. The drug combination of any one of the preceding forms, formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation.
26. The drug combination of any one of the preceding forms, contained in a delivery vehicle, product of manufacture, container, syringe, device or bag.
27. The drug combination of any one of the preceding forms, initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In some embodiments, forms of the invention may include the following.

1. Use of a drug combination, comprising:

at least one potassium-competitive acid blocker (PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

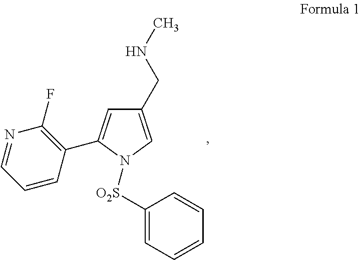 [SMILES: CNCc1cc(-c2cccnc2F)n(S(=O)(=O)c2ccccc2)c1]

a compound having the structure of Formula 2, or equivalent,

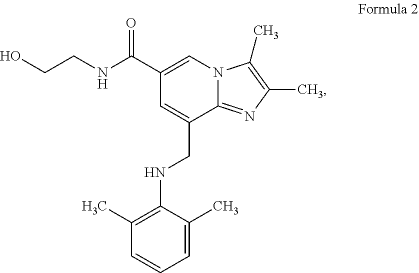 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent,
   in the manufacture of a medicament for treating, ameliorating, reversing and/or preventing (acting as a prophylaxis) a Helicobacter pylori (H. pylori) infection in an individual in need thereof.
   2. The use of form 1, wherein the drug combination further comprises an antimicrobial or antibiotic composition comprising or consisting of:

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is formulated for administration to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally formulated for administration to the individual in need thereof at a dose of between about 10 mg to about 450 mg bid or tid;

(c) a clarithromycin, optionally BIAXIN™,
optionally formulated for administration to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally formulated for administration to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally formulated for administration to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally formulated for administration to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally formulated for administration to the individual in need thereof at a daily dose of between about 50 mg to 3 grams;

(h) a levofloxacin, optionally LEVAQUIN™, TAVANIC™, IQUIX™,
optionally formulated for administration to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally formulated for administration to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally formulated for administration to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally formulated for administration to the individual in need thereof at a dose of between about 15 mg to about 50 mg per day, or at about 25 to 5000 mg per day;

(l) a furazolidone, optionally FUROXONE™, DEPENDAL-M™,
optionally formulated for administration to the individual in need thereof at a dose of between about 5 to about 6000 mg/d;

(m) a rifabutin, optionally MYCOBUTIN™,
optionally formulated for administration to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally formulated for administration to the individual in need thereof at a dose of between about 50 to 2000 mg/day or between about 50 to 2000 mg bid;

(o) a furazolidine, optionally FUROXONE™, DEPENDAL-M™,
optionally formulated for administration to the individual in need thereof at a dose of between about 5 mg to about 6000 mg per day;

(p) a bismuth, bismuth salicylate or bismuth subsalicylate, optionally PEPTO-BISMOL™,
optionally formulated for administration to the individual in need thereof at a dose of between about 100 mg to about 4000 mg per day;

(q) tetracycline, optionally SUMYCIN™, TETRACYN™, PANMYCIN™,
optionally formulated for administration to the individual in need thereof at a dose of between about 60 mg to 5000 mg per day;

(r) a doxycycline, optionally DORYZ™, DOXYHEXA™ L, DOXYLIN™,
optionally formulated for administration to the individual in need thereof at a dose of between about 60 mg to 5000 mg per day;

(s) a minocycline, optionally MINOCIN™, MINOCYCIN™, AKAMIN™,
optionally formulated for administration to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is formulated for administration at a dose of between about 500 mg to 5 gm per day, or 250 mg to 6 gm per day.

3. The use of form 1, wherein the medicament is formulated for administration in combination with an antimicrobial or antibiotic composition comprising or consisting of:

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is formulated for administration to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally formulated for administration to the individual in need thereof at a dose of between about 10 mg to about 450 mg bid or tid;

(c) a clarithromycin, optionally BIAXIN™,
optionally formulated for administration to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally formulated for administration to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally formulated for administration to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally formulated for administration to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally formulated for administration to the individual in need thereof at a daily dose of between about 50 mg to 3 grams;

(h) a levofloxacin, optionally LEVAQUIN™, TAVANIC™, IQUIX™,
optionally formulated for administration to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally formulated for administration to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally formulated for administration to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally formulated for administration to the individual in need thereof at a dose of between about 15 mg to about 50 mg per day, or at about 25 to 5000 mg per day;

(l) a furazolidone, optionally FUROXONE™, DEPENDAL-M™,
optionally formulated for administration to the individual in need thereof at a dose of between about 5 to about 6000 mg/d;

(m) a rifabutin, optionally MYCOBUTIN™,
optionally formulated for administration to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally formulated for administration to the individual in need thereof at a dose of between about 50 to 2000 mg/day or between about 50 to 2000 mg bid;

(o) a furazolidine, optionally FUROXONE™, DEPENDAL-M™,
optionally formulated for administration to the individual in need thereof at a dose of between about 5 mg to about 6000 mg per day;

(p) a bismuth, bismuth salicylate or bismuth subsalicylate, optionally PEPTO-BISMOL™,
optionally formulated for administration to the individual in need thereof at a dose of between about 100 mg to about 4000 mg per day;

(q) tetracycline, optionally SUMYCIN™, TETRACYN™, PANMYCIN™,
optionally formulated for administration to the individual in need thereof at a dose of between about 60 mg to 5000 mg per day;

(r) a doxycycline, optionally DORYZ™, DOXYHEXA™L, DOXYLIN™,
optionally formulated for administration to the individual in need thereof at a dose of between about 60 mg to 5000 mg per day;

(s) a minocycline, optionally MINOCIN™, MINOCYCIN™, AKAMIN™,
optionally formulated for administration to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is formulated for administration at a dose of between about 500 mg to 5 gm per day, or 250 mg to 6 gm per day.

4. The use of form 1 or form 2, wherein the drug combination comprises or consists of:

(1) tegoprazan, revaprazan, linaprazan or soraprazan; and, amoxicillin or amoxicillin/clavulanic acid;

(2) tegoprazan, revaprazan, linaprazan or soraprazan; and, clarithromycin;

(3) tegoprazan, revaprazan, linaprazan or soraprazan; clarithromycin; and, amoxicillin or amoxicillin/clavulanic acid;

(4) tegoprazan, revaprazan, linaprazan or soraprazan; and, rifabutin;

(5) tegoprazan, revaprazan, linaprazan or soraprazan; clarithromycin; and, rifabutin;

(6) tegoprazan, revaprazan, linaprazan or soraprazan; amoxicillin or amoxicillin/clavulanic acid; and, rifabutin;

(7) tegoprazan, revaprazan, linaprazan or soraprazan; amoxicillin or amoxicillin/clavulanic acid; clarithromycin; and, rifabutin;

(8) tegoprazan, revaprazan, linaprazan or soraprazan; and, levofloxacin;

(9) tegoprazan, revaprazan, linaprazan or soraprazan; clarithromycin; and, levofloxacin;

(10) tegoprazan, revaprazan, linaprazan or soraprazan; amoxicillin or amoxicillin/clavulanic acid; and, levofloxacin;

(12) tegoprazan, revaprazan, linaprazan or soraprazan; amoxicillin or amoxicillin/clavulanic acid; clarithromycin; and, levofloxacin;

(13) tegoprazan, revaprazan, linaprazan or soraprazan; amoxicillin or amoxicillin/clavulanic acid; rifabutin; and, levofloxacin;

(14) tegoprazan, revaprazan, linaprazan or soraprazan; amoxicillin or amoxicillin/clavulanic acid; rifabutin; clarithromycin; and, levofloxacin;

(15) tegoprazan, revaprazan, linaprazan or soraprazan; amoxicillin or amoxicillin/clavulanic acid; rifabutin; and, tetracycline;

(16) tegoprazan, revaprazan, linaprazan or soraprazan; amoxicillin or amoxicillin/clavulanic acid; rifabutin; clarithromycin; and, tetracycline;

(17) tegoprazan, revaprazan, linaprazan or soraprazan; and, tetracycline;

(18) tegoprazan, revaprazan, linaprazan or soraprazan; clarithromycin; and, tetracycline;

(19) tegoprazan, revaprazan, linaprazan or soraprazan; and, metronidazole or tinidazole;

(20) tegoprazan, revaprazan, linaprazan or soraprazan; clarithromycin; and, metronidazole or tinidazole;

(21) tegoprazan, revaprazan, linaprazan or soraprazan; and, a bismuth, bismuth salicylate or bismuth subsalicylate;

(22) tegoprazan, revaprazan, linaprazan or soraprazan; clarithromycin; and, a bismuth, bismuth salicylate or bismuth subsalicylate;

(23) tegoprazan, revaprazan, linaprazan or soraprazan; tetracycline; and, a bismuth, bismuth salicylate or bismuth subsalicylate;

(24) tegoprazan, revaprazan, linaprazan or soraprazan; tetracycline; clarithromycin; and, a bismuth, bismuth salicylate or bismuth subsalicylate;

(25) tegoprazan, revaprazan, linaprazan or soraprazan; tetracycline; a bismuth, bismuth salicylate or bismuth subsalicylate; and, metronidazole or tinidazole;

(26) tegoprazan, revaprazan, linaprazan or soraprazan; tetracycline; a bismuth, bismuth salicylate or bismuth subsalicylate; clarithromycin; and, metronidazole or tinidazole;

(27) tegoprazan, revaprazan, linaprazan or soraprazan; tetracycline; and, metronidazole or tinidazole;

(28) tegoprazan, revaprazan, linaprazan or soraprazan; tetracycline; clarithromycin; and, metronidazole or tinidazole;

(29) tegoprazan, revaprazan, linaprazan or soraprazan; and, ampicillin;

(30) tegoprazan, revaprazan, linaprazan or soraprazan; clarithromycin; and, ampicillin;

(31) tegoprazan, revaprazan, linaprazan or soraprazan; tetracycline; metronidazole or tinidazole; and, ampicillin;

(32) tegoprazan, revaprazan, linaprazan or soraprazan; tetracycline; metronidazole or tinidazole; clarithromycin; and, ampicillin;

(33) tegoprazan, revaprazan, linaprazan or soraprazan; tetracycline; and, ampicillin;

(34) tegoprazan, revaprazan, linaprazan or soraprazan; tetracycline; clarithromycin; and, ampicillin;

(35) tegoprazan, revaprazan, linaprazan or soraprazan; and, rifabutin; tegoprazan, revaprazan, linaprazan or soraprazan; clarithromycin; and, rifabutin;

(36) tegoprazan, revaprazan, linaprazan or soraprazan; and, rifampicin;

(37) tegoprazan, revaprazan, linaprazan or soraprazan; clarithromycin; and, rifampicin;

(38) tegoprazan, revaprazan, linaprazan or soraprazan; rifabutin; and, rifampicin;

(39) tegoprazan, revaprazan, linaprazan or soraprazan; rifabutin; clarithromycin; and, rifampicin;

(40) tegoprazan, revaprazan, linaprazan or soraprazan; rifabutin; tetracycline, and, rifampicin;

(41) tegoprazan, revaprazan, linaprazan or soraprazan; rifabutin; tetracycline, clarithromycin; and, rifampicin;

(42) tegoprazan, revaprazan, linaprazan or soraprazan; and, clarithromycin; tegoprazan, revaprazan, and, clarithromycin;

(43) tegoprazan, revaprazan, linaprazan or soraprazan; rifabutin; and, clarithromycin;

(44) tegoprazan, revaprazan, linaprazan or soraprazan; and, clarithromycin;

(45) tegoprazan, revaprazan, linaprazan or soraprazan; rifampicin; and, clarithromycin;

(46) rifampicin; and, clarithromycin;

(47) tegoprazan, revaprazan, linaprazan or soraprazan; and, azithromycin;

(48) tegoprazan, revaprazan, linaprazan or soraprazan; clarithromycin; and, azithromycin;

(49) tegoprazan, revaprazan, linaprazan or soraprazan; rifampicin; and, azithromycin;

(50) tegoprazan, revaprazan, linaprazan or soraprazan; clarithromycin; rifampicin; and, azithromycin;

(51) tegoprazan, revaprazan, linaprazan or soraprazan; rifabutin; and, azithromycin;

(52) tegoprazan, revaprazan, linaprazan or soraprazan; rifabutin; clarithromycin; and, azithromycin;

(53) tegoprazan, revaprazan, linaprazan or soraprazan; rifabutin; and, a vonoprazan or a vonoprazan fumarate;

(54) tegoprazan, revaprazan, linaprazan or soraprazan; rifabutin; clarithromycin; and, a vonoprazan or a vonoprazan fumarate;

(55) tegoprazan, revaprazan, linaprazan or soraprazan; rifabutin; and, metronidazole;

(55) tegoprazan, revaprazan, linaprazan or soraprazan; rifabutin; clarithromycin; and, metronidazole;

(56) tegoprazan, revaprazan, linaprazan or soraprazan; rifabutin; and, tinidazole;

(57) tegoprazan, revaprazan, linaprazan or soraprazan; rifabutin; clarithromycin; and, tinidazole;

(58) tegoprazan, revaprazan, linaprazan or soraprazan; rifabutin; and, nitazoxanide;

(59) tegoprazan, revaprazan, linaprazan or soraprazan; rifabutin; clarithromycin; and, nitazoxanide;

(60) tegoprazan, revaprazan, linaprazan or soraprazan; rifabutin; and, furazolidine;

(61) tegoprazan, revaprazan, linaprazan or soraprazan; rifabutin; clarithromycin; and, furazolidine;

(62) tegoprazan, revaprazan, linaprazan or soraprazan; and, ciprofloxacin;

(63) tegoprazan, revaprazan, linaprazan or soraprazan; clarithromycin; and, ciprofloxacin;

(64) tegoprazan, revaprazan, linaprazan or soraprazan; rifabutin; and, ciprofloxacin;

(65) tegoprazan, revaprazan, linaprazan or soraprazan; rifabutin; clarithromycin; and, ciprofloxacin

(66) tegoprazan, revaprazan, linaprazan or soraprazan; and, doxycycline or minocycline; or

(67) tegoprazan, revaprazan, linaprazan or soraprazan; clarithromycin; and, doxycycline or minocycline.

5. The use of any one of the preceding forms, wherein the drug combination comprises a potassium competitive acid blocker and amoxicillin.
6. The use of form 5, wherein the amoxicillin is administered in an amount of at least 3 g/day.
7. The use of any one of the preceding forms, wherein the drug combination comprises a potassium competitive acid blocker, amoxicillin, and clarithromycin.
7. The use of any one of the preceding forms, wherein the drug combination comprises a potassium competitive acid blocker, amoxicillin, and a rifamycin derivative.
8. The use of form 7, wherein the rifamycin derivative is rifabutin.
9. The use of any one of forms 5 to 8, wherein the potassium competitive acid blocker is selected from the group consisting of tegoprazan, revaprazan, and linaprazan.
10. The use of any one of the preceding forms, wherein the drug combination is:

(i) tegoprazan, amoxicillin, and clarithromycin;

(ii) tegoprazan and amoxicillin; or

(iii) revaprazan, amoxicillin, and rifabutin.

11. The use of any one of forms 1 to 4, wherein the drug combination comprises a potassium competitive acid blocker, a tetracycline antibiotic, a nitroimidazole antibiotic, and a bismuth salt.
12. The use of form 11, wherein the drug combination comprises linaprazan, tetracycline, tinidazole, and bismuth subsalicylate.
13. The use of any one of the preceding forms, wherein the medicament is formulated for administration to the individual in need thereof for about 2, 3, 4, 5, 6, 7, 8, 9, 10, 11, 12, 13 or 14 or more days.
14. The use of any one of forms 2 to 13, wherein the antimicrobial or antibiotic composition is formulated for administration together with, before and/or after the potassium competitive acid blocker, the acid pump blocker or the inhibitor of H+/K+ ATPase.
15. The use of any one of forms 2 to 14, wherein the antimicrobial or antibiotic composition is formulated together with, or separately from, the potassium competitive acid blocker, the acid pump blocker or the inhibitor of H+/K+ ATPase.
16. The use of any one of the preceding forms, wherein the drug combination is contained in or comprises one or more of a formulation, a pharmaceutical preparation or a pharmaceutical composition.
17. The use of any one of the preceding forms, wherein any one or several of the potassium competitive acid blocker or the acid pump blocker, or the inhibitor of H+/K+ ATPase, or the antimicrobial or antibiotic composition is formulated for administration to the individual in need thereof:

(a) at a unit dosage of between about 5 mg to about 5000 mg per day, or

(b) in a unit dosage form of between about 10 mg and 200 mg, or between about between about 40 mg and 100 mg, or between about between about 100 mg and 500 mg, or between about between about 500 mg and 1000 mg, or is about 10, 20, 30, 40, 50, 60, 70, 75, 80, 90 or 100 mg per unit dose,

which optionally is formulated for administration in a once a day, bid or tid, or a four times a day, five times a day or six times a day or more, regimen.

18. The use of any one of the preceding forms, wherein the medicament is formulated as a chewable delivery vehicle, a gum, a gummy, a candy, a lozenge, an ice cream or an ice, or a yogurt.
19. The use of any one of the preceding forms, wherein the medicament further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
20. The use of any one of the preceding forms, wherein the medicament further comprises a preservative, a benzoic acid or a potassium sorbate.
21. The use of any one of the preceding forms, wherein the medicament further comprises at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep. fecalis and equivalents.
22. The use of any one of the preceding forms, wherein the medicament further comprises at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
23. The use of any one of the preceding forms, wherein the medicament further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
24. The use of any one of the preceding forms, wherein the medicament further comprises at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™), atorvastatin (optionally LIPITOR™), pravastatin (optionally PRAVACHOL™) fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
25. The use of any one of the preceding forms, wherein the medicament is formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation.
26. The use of any one of the preceding forms, wherein the medicament is contained in a delivery vehicle, product of manufacture, container, syringe, device or bag.
27. The use of any one of the preceding forms, wherein the medicament is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation

In alternative embodiments, provided are methods for treating, ameliorating, reversing and/or preventing (acting as a prophylaxis) a Helicobacter pylori (H. pylori) infection in an individual in need thereof, comprising:

administering to the individual in need thereof a therapeutic or a drug, or a therapeutic or a drug combination, comprising:

at least one potassium-competitive acid blocker (or PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

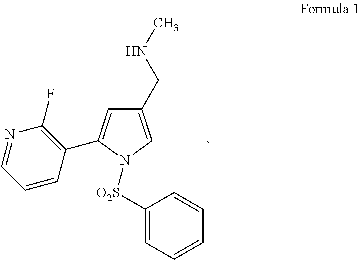 [SMILES: CNCc1cc(-c2cccnc2F)n(S(=O)(=O)c2ccccc2)c1]

a compound having the structure of Formula 2, or equivalent,

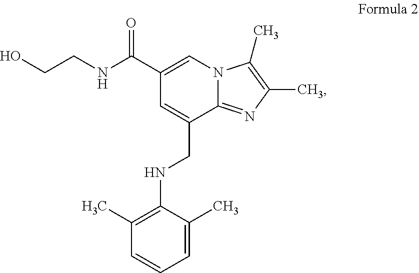 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent.

In alternative embodiments, methods as provided herein further comprise co-administering with the potassium competitive acid blocker or the acid pump blocker, or the inhibitor of H+/K+ ATPase, an antimicrobial or antibiotic drug or composition comprising or consisting of:

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally administered to the individual in need thereof at a dose of between about 10 mg to about 450 mg bid or tid;

(c) a clarithromycin, optionally BIAXIN™,
optionally administered to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally administered to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally administered to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally administered to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally administered to the individual in need thereof at a daily dose of between about 50 mg to 3 grams;

(h) a levofloxacin, optionally LEVAQUIN™, TAVANIC™, IQUIX™,
optionally administered to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally administered to the individual in need thereof at a dose of between about 10 mg to about 2500 mg twice daily (bid) or three times daily (tid);

(j) a moxifloxacin, optionally AVELOX™, VIGAMOX™, MOXEZA™
optionally administered to the individual in need thereof at a dose of between about 10 mg to about 2500 mg per day,

(k) a TG44, or a [4-methylbenzyl 4′-[trans-4-(guanidine-methyl) cyclohexyl carbonyloxy] biphenyl-4-carboxylate monohydrochloride, or CAS registry number 178748-55-5,
optionally administered to the individual in need thereof at a dose of between about 15 mg to about 50 mg per day, or at about 25 to 5000 mg per day;

(l) a furazolidone, optionally FUROXONE™, DEPENDAL-M™,
optionally administered to the individual in need thereof at a dose of between about 5 to about 6000 mg/d;

(m) a rifabutin, optionally MYCOBUTIN™,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally administered to the individual in need thereof at a dose of between about 50 to 2000 mg/day or between about 50 to 2000 mg bid;

(o) a furazolidine, optionally FUROXONE™, DEPENDAL-M™,
optionally administered to the individual in need thereof at a dose of between about 5 mg to about 6000 mg per day;

(p) a bismuth, bismuth salicylate or bismuth subsalicylate, optionally PEPTO-BISMOL™,
optionally administered to the individual in need thereof at a dose of between about 100 mg to about 4000 mg per day;

(q) tetracycline, optionally SUMYCIN™, TETRACYN™, PANMYCIN™,
optionally administered to the individual in need thereof at a dose of between about 60 mg to 5000 mg per day;

(r) a doxycycline, optionally DORYZ™, DOXYHEXA™L, DOXYLIN™
optionally administered to the individual in need thereof at a dose of between about 60 mg to 5000 mg per day;

(s) a minocycline, optionally MINOCIN™, MINOCYCIN™, AKAMIN™,
optionally administered to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is administered at a dose of between about 500 mg to 5 gm per day, or 250 mg to 6 gm per day.

In alternative embodiments, a therapeutic or drug combination is or comprises, or a therapeutic or drug combination used in a method as provided herein, is or comprises:

(1) tegoprazan, revaprazan, linaprazan or soraprazan; and, amoxicillin or amoxicillin/clavulanic acid;

(2) tegoprazan, revaprazan, linaprazan or soraprazan; and, clarithromycin;

(3) tegoprazan, revaprazan, linaprazan or soraprazan; clarithromycin; and, amoxicillin or amoxicillin/clavulanic acid;

(4) tegoprazan, revaprazan, linaprazan or soraprazan; and, rifabutin;

(5) tegoprazan, revaprazan, linaprazan or soraprazan; clarithromycin; and, rifabutin;

(6) tegoprazan, revaprazan, linaprazan or soraprazan; amoxicillin or amoxicillin/clavulanic acid; and, rifabutin;

(7) tegoprazan, revaprazan, linaprazan or soraprazan; amoxicillin or amoxicillin/clavulanic acid; clarithromycin; and, rifabutin;

(8) tegoprazan, revaprazan, linaprazan or soraprazan; and, levofloxacin;

(9) tegoprazan, revaprazan, linaprazan or soraprazan; clarithromycin; and, levofloxacin;

(10) tegoprazan, revaprazan, linaprazan or soraprazan; amoxicillin or amoxicillin/clavulanic acid; and, levofloxacin;

(12) tegoprazan, revaprazan, linaprazan or soraprazan; amoxicillin or amoxicillin/clavulanic acid; clarithromycin; and, levofloxacin;

(13) tegoprazan, revaprazan, linaprazan or soraprazan; amoxicillin or amoxicillin/clavulanic acid; rifabutin; and, levofloxacin;

(14) tegoprazan, revaprazan, linaprazan or soraprazan; amoxicillin or amoxicillin/clavulanic acid; rifabutin; clarithromycin; and, levofloxacin;

(15) tegoprazan, revaprazan, linaprazan or soraprazan; amoxicillin or amoxicillin/clavulanic acid; rifabutin; and, tetracycline;

(16) tegoprazan, revaprazan, linaprazan or soraprazan; amoxicillin or amoxicillin/clavulanic acid; rifabutin; clarithromycin; and, tetracycline;

(17) tegoprazan, revaprazan, linaprazan or soraprazan; and, tetracycline;

(18) tegoprazan, revaprazan, linaprazan or soraprazan; clarithromycin; and, tetracycline;

(19) tegoprazan, revaprazan, linaprazan or soraprazan; and, metronidazole or tinidazole;

(20) tegoprazan, revaprazan, linaprazan or soraprazan; clarithromycin; and, metronidazole or tinidazole;

(21) tegoprazan, revaprazan, linaprazan or soraprazan; and, a bismuth, bismuth salicylate or bismuth subsalicylate;

(22) tegoprazan, revaprazan, linaprazan or soraprazan; clarithromycin; and, a bismuth, bismuth salicylate or bismuth subsalicylate;

(23) tegoprazan, revaprazan, linaprazan or soraprazan; tetracycline; and, a bismuth, bismuth salicylate or bismuth subsalicylate;

(24) tegoprazan, revaprazan, linaprazan or soraprazan; tetracycline; clarithromycin; and, a bismuth, bismuth salicylate or bismuth subsalicylate;

(25) tegoprazan, revaprazan, linaprazan or soraprazan; tetracycline; a bismuth, bismuth salicylate or bismuth subsalicylate; and, metronidazole or tinidazole;

(26) tegoprazan, revaprazan, linaprazan or soraprazan; tetracycline; a bismuth, bismuth salicylate or bismuth subsalicylate; clarithromycin; and, metronidazole or tinidazole;

(27) tegoprazan, revaprazan, linaprazan or soraprazan; tetracycline; and, metronidazole or tinidazole;

(28) tegoprazan, revaprazan, linaprazan or soraprazan; tetracycline; clarithromycin; and, metronidazole or tinidazole;

(29) tegoprazan, revaprazan, linaprazan or soraprazan; and, ampicillin;

(30) tegoprazan, revaprazan, linaprazan or soraprazan; clarithromycin; and, ampicillin;

(31) tegoprazan, revaprazan, linaprazan or soraprazan; tetracycline; metronidazole or tinidazole; and, ampicillin;

(32) tegoprazan, revaprazan, linaprazan or soraprazan; tetracycline; metronidazole or tinidazole; clarithromycin; and, ampicillin;

(33) tegoprazan, revaprazan, linaprazan or soraprazan; tetracycline; and, ampicillin;

(34) tegoprazan, revaprazan, linaprazan or soraprazan; tetracycline; clarithromycin; and, ampicillin;

(35) tegoprazan, revaprazan, linaprazan or soraprazan; and, rifabutin; tegoprazan, revaprazan, linaprazan or soraprazan; clarithromycin; and, rifabutin;

(36) tegoprazan, revaprazan, linaprazan or soraprazan; and, rifampicin;

(37) tegoprazan, revaprazan, linaprazan or soraprazan; clarithromycin; and, rifampicin;

(38) tegoprazan, revaprazan, linaprazan or soraprazan; rifabutin; and, rifampicin;

(39) tegoprazan, revaprazan, linaprazan or soraprazan; rifabutin; clarithromycin; and, rifampicin;

(40) tegoprazan, revaprazan, linaprazan or soraprazan; rifabutin; tetracycline, and, rifampicin;

(41) tegoprazan, revaprazan, linaprazan or soraprazan; rifabutin; tetracycline, clarithromycin; and, rifampicin;

(42) tegoprazan, revaprazan, linaprazan or soraprazan; and, clarithromycin; tegoprazan, revaprazan, and, clarithromycin;

(43) tegoprazan, revaprazan, linaprazan or soraprazan; rifabutin; and, clarithromycin;

(44) tegoprazan, revaprazan, linaprazan or soraprazan; and, clarithromycin;

(45) tegoprazan, revaprazan, linaprazan or soraprazan; rifampicin; and, clarithromycin;

(46) rifampicin; and, clarithromycin;

(47) tegoprazan, revaprazan, linaprazan or soraprazan; and, azithromycin;

(48) tegoprazan, revaprazan, linaprazan or soraprazan; clarithromycin; and, azithromycin;

(49) tegoprazan, revaprazan, linaprazan or soraprazan; rifampicin; and, azithromycin;

(50) tegoprazan, revaprazan, linaprazan or soraprazan; clarithromycin; rifampicin; and, azithromycin;

(51) tegoprazan, revaprazan, linaprazan or soraprazan; rifabutin; and, azithromycin;

(52) tegoprazan, revaprazan, linaprazan or soraprazan; rifabutin; clarithromycin; and, azithromycin;

(53) tegoprazan, revaprazan, linaprazan or soraprazan; rifabutin; and, a vonoprazan or a vonoprazan fumarate;

(54) tegoprazan, revaprazan, linaprazan or soraprazan; rifabutin; clarithromycin; and, a vonoprazan or a vonoprazan fumarate;

(55) tegoprazan, revaprazan, linaprazan or soraprazan; rifabutin; and, metronidazole;

(55) tegoprazan, revaprazan, linaprazan or soraprazan; rifabutin; clarithromycin; and, metronidazole;

(56) tegoprazan, revaprazan, linaprazan or soraprazan; rifabutin; and, tinidazole;

(57) tegoprazan, revaprazan, linaprazan or soraprazan; rifabutin; clarithromycin; and, tinidazole;

(58) tegoprazan, revaprazan, linaprazan or soraprazan; rifabutin; and, nitazoxanide;

(59) tegoprazan, revaprazan, linaprazan or soraprazan; rifabutin; clarithromycin; and, nitazoxanide;

(60) tegoprazan, revaprazan, linaprazan or soraprazan; rifabutin; and, furazolidine;

(61) tegoprazan, revaprazan, linaprazan or soraprazan; rifabutin; clarithromycin; and, furazolidine;

(62) tegoprazan, revaprazan, linaprazan or soraprazan; and, ciprofloxacin;

(63) tegoprazan, revaprazan, linaprazan or soraprazan; clarithromycin; and, ciprofloxacin;

(64) tegoprazan, revaprazan, linaprazan or soraprazan; rifabutin; and, ciprofloxacin;

(65) tegoprazan, revaprazan, linaprazan or soraprazan; rifabutin; clarithromycin; and, ciprofloxacin

(66) tegoprazan, revaprazan, linaprazan or soraprazan; and, doxycycline or minocycline; or

(67) tegoprazan, revaprazan, linaprazan or soraprazan; clarithromycin; and, doxycycline or minocycline.

In alternative embodiments, the drug or therapeutic combination is administered to the individual in need thereof for about 2, 3, 4, 5, 6, 7, 8, 9, 10, 11, 12, 13 or 14 or more days.

In alternative embodiments, any one, two, three or four of more drugs or therapeutic combination as provide herein is/are administered together with, before and/or after administration of the potassium competitive acid blocker, the acid pump blocker or the inhibitor of H+/K+ ATPase; or, are formulated together, or separately from, with the potassium competitive acid blocker, the acid pump blocker or the inhibitor of H+/K+ ATPase.

In alternative embodiments, the drug or therapeutic combination is contained in or comprises one or more of a formulation, a pharmaceutical preparation or a pharmaceutical composition.

In alternative embodiments of methods as provided herein, any one or several of the drugs is administered to the individual in need thereof:

(a) at a unit dosage of between about 5 mg to about 5000 mg per day, or

(b) in a unit dosage form of between about 10 mg and 200 mg, or between about between about 40 mg and 100 mg, or between about between about 100 mg and 500 mg, or between about between about 500 mg and 1000 mg, or is about 10, 20, 30, 40, 50, 60, 70, 75, 80, 90 or 100 mg per unit dose,

which optionally can be administered once a day, bid or tid, or a four times a day, five times a day or six times a day or more, regimen.

In alternative embodiments, therapeutic combinations or formulations, or pharmaceuticals or the pharmaceutical preparations as provided herein or as used in methods as provided herein are formulated as a chewable delivery vehicle, a gum, a gummy, a candy, a lozenge, an ice cream or an ice, or a yogurt.

In alternative embodiments, therapeutic combinations or formulations, or pharmaceuticals or the pharmaceutical preparations as provided herein or as used in methods as provided herein further comprise a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof; or, further comprise a preservative, a benzoic acid or a potassium sorbate; or, further comprise, or have added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep. fecalis and equivalents; or, further comprise, or have added to: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch; or, further comprise an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient; or, further comprise, or have added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™) atorvastatin (optionally LIPITOR™), pravastatin (optionally PRAVACHOL™), fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, therapeutic combinations or formulations, or pharmaceuticals or the pharmaceutical preparations as provided herein or as used in methods as provided herein are formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation.

In alternative embodiments, therapeutic combinations or formulations, or pharmaceuticals or the pharmaceutical preparations as provided herein or as used in methods as provided herein are contained in a delivery vehicle, product of manufacture, container, syringe, device or bag.

In alternative embodiments, therapeutic combinations or formulations, or pharmaceuticals or the pharmaceutical preparations as provided herein or as used in methods as provided herein are initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In alternative embodiments, provided are therapeutic combinations comprising:

(a) a therapeutic or a drug combination as used in any method as provided herein; or

(b)

[symbol]

(i) a therapeutic or a drug combination comprising at least one potassium-competitive acid blocker (or PCAB) or acid pump blocker (or acid pump antagonist (APA)), or at least one inhibitor of H+/K+ ATPase,
wherein optionally the at least one potassium competitive acid blocker is or comprises:
   tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
   revaprazan (optionally REVANEX™) or N-(4-fluorophenyl)-4,5-dimethyl-6-(1-methyl-1,2,3,4-tetrahydroisoquinolin-2-yl)pyrimidin-2-amine hydrochloride or equivalent,
   linaprazan, or 8-[(2,6-dimethylphenyl)methylamino]-N-(2-hydroxyethyl)-2,3-dimethylimidazo[1,2-a]pyridine-6-carboxamide, or equivalent,
   a compound having the structure of Formula 1, or equivalent,

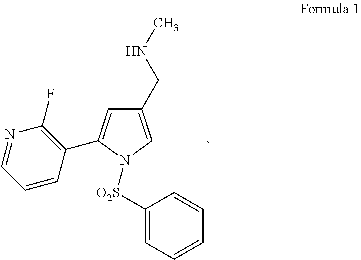 [SMILES: CNCc1cc(-c2cccnc2F)n(S(=O)(=O)c2ccccc2)c1]

a compound having the structure of Formula 2, or equivalent,

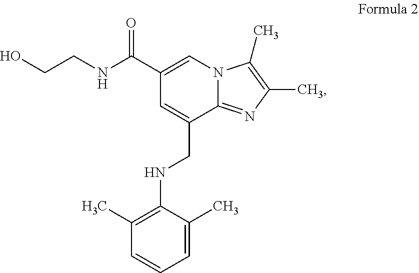 [SMILES: Cc1cccc(C)c1NCc1cc(C(=O)NCCO)cn2c(C)c(C)nc12]

and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[1,2-h][1,7]naphthyridin-8-ol, or equivalent; and

(ii)

[symbol]

(a) at least one penicillin or a penicillin derivative or beta-lactamase inhibitor,

wherein optionally the at least one penicillin derivative or beta-lactamase inhibitor comprises: an amoxicillin; a clavulanate, potassium clavulanate, or clavulanic acid; an ampicillin (optionally PRINCIPEN™); a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav, or is optionally ACTIMOXI™, ALPHAMOX™, AMOCLA™, TYCIL™, AMOXIL™, TRIMOX™.
and optionally the potassium clavulanate or clavulanic acid is formulated with ticarcillin (optionally TIMENTIN™),
and optionally the ampicillin is formulated with sulbactam (optionally UNASYN™);
and optionally the piperacillin is formulated with tazobactam (optionally TAZOCIN™ or ZOSYN™);

(b) a rifampicin or rifampin, optionally RIFADIN™,
optionally administered to the individual in need thereof at a dose of between about 10 mg to about 450 mg bid or tid;

(c) a clarithromycin, optionally BIAXIN™,
optionally administered to the individual in need thereof at a dose of between about 10 mg to about 3 g twice daily (bid) or tid, or at a dose of between about 500 mg to 5 gm per day,

(d) an azithromycin (optionally ZITHROMAZ™, AZITHROCIN™)
optionally administered to the individual in need thereof at a dose of between about 100 mg to 3 grams bid, or once a day, or every second or third day;

(e) a vonoprazan or a vonoprazan fumarate, or a 5-(2-fluorophenyl)-1-(pyridin-3-ylsulfonyl)-1H-pyrrol-3-yl)-N-methylmethanamine monofumarate, or a 1-(5-(2-fluorophenyl)-1-(pyridin-3-ylsulfonyl)-1H-pyrrol-3-yl)-N-methyl-methanamine fumarate), optionally TAKECAB™,
optionally administered to the individual in need thereof at a dose range of between about 1 mg to about 100 mg twice daily (bid), or for about 10 mg to 25 mg or more twice daily (bid);

(f) a metronidazole, optionally FLAGYL™, METRO™,
optionally administered to the individual in need thereof at a dose of between about 10 mg to 20 mg, to about 1000 mg, three times daily (tid), or at a dose range of between about 10 to 2000 mg or more three times daily (tid);

(g) a tinidazole, optionally FASIGYN™, SIMPLOTAN™, TINDAMAX™, SPORINEX™,
optionally administered to the individual in need thereof at a daily dose of between about 50 mg to 3 grams;

(h) a levofloxacin, optionally LEVAQUIN™, TAVANIC™, IQUIX™,
optionally administered to the individual in need thereof at a dose of between about 10 mg to about 5000 mg twice daily (bid) or three times daily (tid);

(i) a ciprofloxacin, optionally CILOXAN™, CIPRO™, NEOFLOXIN™,
optionally administered to the individual in need thereof at a dose of between about 10 mg to about 2500 mg twice daily (bid) or three times daily (tid);

(j) a moxifloxacin, optionally AVELOX™, VIGAMOX™, MOXEZA™,
optionally administered to the individual in need thereof at a dose of between about 10 mg to about 2500 mg per day,

(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally administered to the individual in need thereof at a dose of between about 15 mg to about 50 mg per day, or at about 25 to 5000 mg per day;

(l) a furazolidone, optionally FUROXONE™, DEPENDAL-M™,
optionally administered to the individual in need thereof at a dose of between about 5 to about 6000 mg/d;

(m) a rifabutin, optionally MYCOBUTIN™,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,

(n) a nitazoxanide, optionally ALINIA™, NIZONIDE™,
optionally administered to the individual in need thereof at a dose of between about 50 to 2000 mg/day or between about 50 to 2000 mg bid;

(o) a furazolidine, optionally FUROXONE™, DEPENDAL-M™,
optionally administered to the individual in need thereof at a dose of between about 5 mg to about 6000 mg per day;

(p) a bismuth, bismuth salicylate or bismuth subsalicylate, optionally PEPTO-BISMOL™,
optionally administered to the individual in need thereof at a dose of between about 100 mg to about 4000 mg per day;

(q) tetracycline, optionally SUMYCIN™, TETRACYN™, PANMYCIN™,
optionally administered to the individual in need thereof at a dose of between about 60 mg to 5000 mg per day;

(r) a doxycycline, optionally DORYZ™, DOXYHEXA™L, DOXYLIN™,
optionally administered to the individual in need thereof at a dose of between about 60 mg to 5000 mg per day;

(s) a minocycline, optionally MINOCIN™, MINOCYCIN™, AKAMIN™,
optionally administered to the individual in need thereof at a dose of between about 60 to about 5000 mg daily;

(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or

(u) any combination of (a) to (t) further comprising a clarithromycin, wherein optionally the clarithromycin is administered at a dose of between about 500 mg to 5 gm per day, or 250 mg to 6 gm per day.

In alternative embodiments, provided are kits or products of manufacture comprising a therapeutic or drug combination as provided herein, or a therapeutic combination as used in a method as provided herein.

In alternative embodiments, provided are use of a therapeutic combination as provided herein, or use of a therapeutic combination as used in a method as provided herein, or a kit or product of manufacture as provided herein, in the manufacture of a medicament.

In alternative embodiments, provided are therapeutic combinations as described herein for use in treating, ameliorating, reversing and/or preventing (acting as a prophylaxis) a Helicobacter pylori (H. pylori) infection in an individual in need thereof.

The details of one or more exemplary embodiments of the invention are set forth in the description below. Other features, objects, and advantages of the invention will be apparent from the description, and from the claims.

All publications, patents, patent applications cited herein are hereby expressly incorporated by reference for all purposes.

In a first aspect, forms of the invention described herein include the following:

### DESCRIPTION OF EMBODIMENTS

In alternative embodiments, provided are therapeutic combinations (including formulations, pharmaceutical preparations or pharmaceutical compositions) using at least one potassium competitive acid blocker or acid pump blocker, or at least one inhibitor of H+/K+ ATPase, for treating, ameliorating, reversing and/or preventing (acting as a prophylaxis) a Helicobacter pylori (H. pylori, or “HP”) infection in an individual in need thereof, including products of manufacture and kits, for practicing methods as provided herein.

In alternative embodiments, therapeutic combinations, formulations, pharmaceutical preparations or pharmaceutical compositions as provided herein use at least one potassium competitive acid blocker or acid pump blocker, or at least one inhibitor of H+/K+ ATPase, to improve efficacy in novel combinations to achieve close to 100% eradication of HP infections; and this significant breakthrough is particularly relevant for those individuals in need thereof who have previously failed eradication of HP, or at least have failed to get any or clinically sufficient resolution of an HP infection and/or symptoms associated with HP infection.

The emergence of the potassium competitive blockers has brought a new dimension to HP therapies. These new acid-lowering medications, can so profoundly reduce acid secretion by parietal calls in the stomach that the pH can rise to 5, 6 or above for prolonged periods. It this rise in the gastric contents pH (or alkalinization) that permits the accompanying antibiotics to better destroy the HP in the mucus and first layers of cells. Efficacy is profoundly elevated making the eradication antibiotic combination therapies work ever so much better.

In alternative embodiments, therapeutic combinations, formulations, pharmaceutical preparations or pharmaceutical compositions as provided herein use the unexpected bactericidal activity of the new compositions mitigating resistance which give new life to otherwise failing of lagging HP eradication protocols, as discussed above.

In alternative embodiments, provided are therapeutic combinations (including formulations, pharmaceutical preparations or pharmaceutical compositions) which use a potassium blocking agent in a dose of between about 1 mg to 5 mg to about 200 mg to 400 mg per day, in conjunction with various other antibiotics or other antimicrobials or drugs. In alternative embodiments, the potassium blocking agent is administered for between about one to 30 days, or longer, for example, the potassium blocking agent is administered for between about one to 30 days at a dosage of between about 5 mg to about 200 mg per day.

In alternative embodiments, provided are therapeutic combinations (including formulations, pharmaceutical preparations or pharmaceutical compositions) which may be more effective than the standard protocols, for example, more effective than using an amoxicillin, clarithromycin or vonoprazan protocol. In alternative embodiments, provided are therapeutic combinations and regimens comprising or consisting of an at least one potassium competitive acid blocker or acid pump blocker, or at least one inhibitor of H+/K+ ATPase that may achieve significantly higher eradication than existing protocols.

In alternative embodiments, therapeutic combination as provided herein, and as used to practice methods as provided herein, are formulated and dosaged for oral administration as a powder, for example, a lyophilized powder, which can be inserted into carriers, for example, capsules, tablets, geltabs, and the like, for example, for administration to adults, infants or children to ingest.

In alternative embodiments, therapeutic combination as provided herein, and as used to practice methods as provided herein, are formulated and dosaged for individuals at an age of 2.5 years or above, where the children are unlikely to be able to swallow a capsule; thus, this provided are additional delivery vehicles, products of manufacture and devices to be combined with formulations as provided herein, for example, powders such as lyophilized powders, for example, lyophilized powder in a storage vehicle, for example, capsules, geltabs and the like; for example, provided are delivery vehicles, products of manufacture and devices manufactured as a container, a kit, a package or a pack of a “device and capsule” together, for example, operably associated such that the container, kit, package or a pack permits individuals, for example, the very young children and the older children (and including disabled or handicapped individuals) to ingest the product, for example, the lyophilized product, from the storage vehicle, for example, capsules, geltabs and the like.

In alternative embodiments, the container, kit, a package or a pack provides the ability of any age child (or disabled or handicapped individual, or any individual) to ingest or swallow the product (for example, a therapeutic combination, a formulation, pharmaceutical preparation or pharmaceutical composition as provided herein) within the storage vehicle (for example, capsule) by “draining”, for example, by puncturing, crushing, twisting or turning the container by hand or a device, or otherwise opening, the storage vehicle using a puncturing, crushing or equivalent device (operably built into the container, kit, package or pack), or by hand motion, for example, by twisting or hand turning (for example, by hand) the container, and thus allowing passage or contact of the contents of the storage vehicle to enter or pass into an ingestible liquid or other edible substance (for example, an ice cream or a yoghurt), which is also contained within the container, kit, package or pack, which can be initially (before the twisting or turning, puncturing, crushing or otherwise opening) in a separate compartment from the storage compartment. This twisting or turning, or puncturing, crushing or otherwise opening of the storage compartment and the passage or contact of the contents of the storage vehicle to the ingestible liquid effectively places the contents of the storage (for example, a powder or freeze-dry comprised of or within a formulation, pharmaceutical preparation or pharmaceutical composition as provided herein) into the ingestible liquid or substance, which can be for example, water, a milk, a yoghurt, an ice cream, a yogurt, a juice (for example, a fruit juice, an apple juice), an apple sauce, or a masking drink. The container, kit, package or pack can be designed as an infant feeding bottle, for example, comprising a nipple or teat for the very young.

In alternative embodiments, this simple twisting or turning, or puncturing or crushing device, allows the storage containers, for example, geltabs or capsules, to be punctured and/or crushed or otherwise “opened”, allowing the contents of the storage container, (for example, a powder or freeze-dry comprised of or within a therapeutic combination, a formulation, pharmaceutical preparation or pharmaceutical composition as provided herein), to fall out in to the liquid or food compartment, for example, to the bottom end of a device or straight into a bottle or a container held underneath or configured to be attached and underneath. For example, in this way a provider, for example, the mother, can purchase a supply of storage containers, for example, geltabs or capsules, convert them as needed into a powder capable of being mixed a liquid of her choice that the child will be ingesting.

In alternative embodiments, for those capable of swallowing tablets, capsules and the like, the storage containers, for example, geltabs, tablets or capsules, are manufactured as enteric coated to bypass the acid of the stomach and bile of the duodenum, such that the storage containers, for example, geltabs, tablets or capsules open (for example, dissolve) in the jejunum or below.

In alternative embodiments, further provided are instructions for use, for example, that when emptied into a drink, providers (for example, the mothers of infants or children) are advised to choose a drink or food that has its own buffering capacity such as flavoured milk, chocolate milk, ice cream, yoghurt, ice blocks, frozen icicles, or simply milk, for example, that is being fed to the infant or child by a bottle, for example, a milk bottle, with a nipple or teat.

In alternative embodiments, storage containers, for example, geltabs, tablets or capsules, or any formulation as provided herein, also comprises an antacid, for example, a calcium carbonate, magnesium hydroxide, propylene glycol alginate and sodium alginate, or the combination of aluminium hydroxide with magnesium trisilicate, magnesium oxide or magnesium carbonate, so that when the storage container is punctured, crushed or otherwise opened and put into contact with the liquid, for example, the feeding bottle, and ingested, there will be greater protection from acid damage.

In alternative embodiments, therapeutic combinations, formulations, pharmaceuticals or pharmaceutical preparations as provided herein are formulated or manufactured as storage vehicles, for example, tablets, geltabs, pills, capsules and the like; and in alternative embodiments, these storage vehicles are contained in, or contained in a kit with, or packaged with, or sold together with, a storage vehicle ‘cracking’, puncturing, or otherwise opening or releasing device (for example, as a powder, for example, as lyophilized material). These can be dispensed together, or configured together, or manufactured together, as a simple way of meeting the needs of both infants, the very young, older children and needful (for example, handicapped) adults; for example, as a powder, for example, as lyophilized material, for example, from their storage vehicles, for example, as encapsulated therapeutic combinations, formulations, pharmaceuticals or pharmaceutical preparations, thus permitting successful clinical administration on a frequent, for example, bid (twice a day), tid (three times a day), or once a day (daily) basis for prolonged periods, for example for up to one to two months, or longer.

### Multicomponent Packaging

Provided are multi-component delivery systems, for example, products of manufacture, comprising for example, formulations, pharmaceutical preparations or pharmaceutical compositions used to practice methods as provided herein, for example, formulated and dosaged for oral administration as a powder, for example, a lyophilized powder, and another component, for example, a liquid; these multi-component delivery systems, for example, products of manufacture, can be designed or manufactured as described for example, in U.S. Pat. Nos. 8,968,717; 8,931,665; 7,861,854; 7,018,089; 6,626,912; and, U.S. Pat. App. Pub nos. 2010/0034574; 2009/0180923; 20090232886; 2008/0160076; 2007/0087048; 2007/0036830; 2007/0074979; 2005/0205438; 2004/0089563.

### Packaging

Provided are compositions, including preparations, formulations and/or kits, comprising combinations of ingredients, for example, therapeutic combinations as described herein. In alternative embodiments, therapeutic combination can be mixed and administered together, or alternatively, they can be an individual member of a packaged combination of ingredients, for example, a liquid component and a solid product component manufactured in a separate compartment, package, kit or container; for example, where all or a subset of the combinations of ingredients are manufactured in a separate compartment, package or container. In alternative aspects, the package, kit or container comprises a blister package, a clamshell, a tray, a shrink wrap and the like.

In one aspect, the package, the kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a formulations, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of formulations, pharmaceutical preparations or pharmaceutical compositions as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, for example, for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (for example, using an AERGO 8 DUO™, SCA Consumer Packaging, Inc., DeKalb Ill.) using regular heat seal tooling. This alternative aspect, using heat seal tooling, can seal common types of thermoformed packaging.

In alternative embodiments, therapeutic combinations, formulations, pharmaceutical preparations or pharmaceutical compositions are formulated, for example, as a powder, for example, as lyophilized material, for example, a lyophilized encapsulated product, for example, for practicing methods as provided herein, can be packaged alone or in combinations, for example, as “blister packages” or as a plurality of packettes, including as lidded blister packages, lidded blister or blister card or packets or packettes, or a shrink wrap.

In alternative embodiments, laminated aluminium foil blister packs are used, for example, for the preparation of therapeutic combinations, formulations, pharmaceutical preparations or pharmaceutical compositions as provided herein. Products or kits comprise an aqueous solution(s) which are dispensed (for example, by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, for example, using hard temper aluminium (for example, alufoil) lidding material. In one aspect, hermetically-sealed high barrier aluminium (for example, alufoil) laminates are used. In one aspect, products of manufacture include kits or blister packs, use foil laminations and strip packs, stick packs, sachets and pouches, peelable and non-peelable laminations combining foil, paper, or film for high barrier packaging.

### Products of Manufacture and Kits

Provided are products of manufacture and kits for practicing methods as provided herein, comprising a therapeutic combination, a formulation, a pharmaceutical preparation or a pharmaceutical composition as provided herein.

In alternative embodiments, multi-component products of manufacture, including kits or blister packs as provided herein, include memory aids to help remind patients when and how to take the therapeutic combination. This safeguards the therapeutic combination's efficacy by protecting each tablet, geltab or pill until it's taken; gives the product or kit portability, makes it easy to take a dose anytime or anywhere.

The invention will be further described with reference to the examples described herein; however, it is to be understood that the invention is not limited to such examples.

A number of embodiments of the invention have been described. Nevertheless, it can be understood that various modifications may be made without departing from the spirit and scope of the invention. Accordingly, other embodiments are within the scope of the following claims.

### EXAMPLES

### Example 1

Four male patients aged 32 y to 64 y were included in a small trial to examine the efficacy of eradicating resistant Helicobacter pylori. They were treated with tegoprazan (two patients with 200 mg tds and two with 400 mg tds) in combination with amoxicillin 500 mg tds and clarithromycin 250 mg tds with minimal adverse effects. The treatment lasted 10 days. On retesting at six weeks with the urea breath test, all four patients had managed to achieve eradication of H. pylori. This indicates that even 200 mg tds can eradicate the resistant infection using this combination. This is in spite of the fact that the patients had previously failed to be cured by standard Nexium-containing therapies administered between two and four times.

### Example 2

An 82 y old female with a chronic gastric lesser curve ulcer with H. pylori infection on endoscopy was originally treated by her attending gastroenterologist—but pain continued and she was referred for second opinion. On her next gastroscopy multiple biopsies were collected but none showed cancer nor dysplasia. Presence of H. pylori was confirmed. Given previous failure when treated with amoxicillin (500 mg tds), rifabutin (150 mg tds) and omeprazole (20 mg tds) (Talicia)—she was treated with revaprazan (150 mg tds) in the place of omeprazole. Pain progressively subsided and a urea breath test at 5 weeks showed no evidence of Helicobacter. She underwent a subsequent gastroscopy 3 months later and H. pylori was absent on histology and urease test, and the gastric ulcer was healed, with minor scarring. The revaprazan helped the antibiotics to work well.

### Example 3

Two patients with chronic H. pylori infection and epigastric pain were found on urea breath test and gastroscopy to be infected but ulcer-free. One had failed ‘Nexium HP 7’ treatment whilst the other had never received H. pylori treatment. Using compounded linaprazan capsules, taken as 100 mg×3/day both patients were treated for 10 days in combination with bismuth subsalicylate 300 mg tds, tetracycline HC1250 mg tds, and tinidazole 250 mg tds (along with the linaprazan). Urea breath test was negative at 4 and 5 weeks respectively in these patients, now cured of the infection and pain free.

### Example 4

A small prospective study was carried out in 6 patients with H. pylori infection referred by their family physicians after they had continued to be infected in spite of an esomeprazole:amoxil:clarithromycin therapy judged by urea breath tests (UBT). All had dyspepsia symptoms. They were investigated by gastroscopy, found to have no stomach cancer nor ulcer but had evidence of H. pylori on histology and UBT. Each was treated using tegoprazan 400 mg tds combined with high dose amoxicillin (1.5 g tds) both for 14 days (double therapy). All 6 were cured of the infection when UBT was repeated at 4-6 weeks. Followed up they remained UBT negative on their second UBT at 3-6 months. This ‘double therapy’ was successful in all.

1. A method for treating, ameliorating, reversing and/or preventing or acting as a prophylaxis for a Helicobacter pylori (H. pylori) infection in an individual in need thereof, comprising:
administering to the individual in need thereof a drug combination, comprising:
at least one potassium-competitive acid blocker (PCAB) or acid pump blocker (or acid pump antagonist (APA)), or at least one inhibitor of H+/K+ ATPase.

2. The method of claim 1, further comprising co-administering with the potassium competitive acid blocker or the acid pump blocker, or the inhibitor of H+/K+ ATPase, an antimicrobial or antibiotic composition comprising:
(a) at least one penicillin or a penicillin derivative or beta-lactamase inhibitor, wherein optionally the at least one penicillin derivative or beta-lactamase inhibitor comprises: an amoxicillin; a clavulanate, potassium clavulanate, or clavulanic acid; an ampicillin; a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination, also known as co-amoxiclav,
and optionally the potassium clavulanate or clavulanic acid is formulated with ticarcillin,
and optionally the ampicillin is formulated with sulbactam;
and optionally the piperacillin is formulated with tazobactam;
(b) a rifampicin or rifampin,
optionally administered to the individual in need thereof at a dose of between about 10 mg to about 450 mg bid or tid;
(c) a clarithromycin,
optionally administered to the individual in need thereof at a dose of between about 10 mg to about 3 g twice daily (bid) or tid, or at a dose of between about 500 mg to 5 gm per day,
(d) an azithromycin,
optionally administered to the individual in need thereof at a dose of between about 100 mg to 3 grams bid, or once a day, or every second or third day;
(e) a vonoprazan or a vonoprazan fumarate, or a 5-(2-fluorophenyl)-1-(pyridin-3-ylsulfonyl)-IH-pyrrol-3-yl)-N-methylmethanamine monofumarate, or a 1-(5-(2-fluorophenyl)-I-(pyridin-3-ylsulfonyl)-IH-pyrrol-3-yl)-N-methyl-methanamine fumarate), optionally TAKECAB™,
optionally administered to the individual in need thereof at a dose range of between about 1 mg to about 100 mg twice daily (bid), or for about 10 mg to 25 mg or more twice daily (bid);
(f) a metronidazole,
optionally administered to the individual in need thereof at a dose of between about 10 mg to 20 mg, to about 1000 mg, three times daily (tid), or at a dose range of between about 10 to 2000 mg or more three times daily (tid);
(g) a tinidazole,
optionally administered to the individual in need thereof at a daily dose of between about 50 mg to 3 grams;
(h) a levofloxacin,
optionally administered to the individual in need thereof at a dose of between about 10 mg to about 5000 mg twice daily (bid) or three times daily (tid);
(i) a ciprofloxacin,
optionally administered to the individual in need thereof at a dose of between about 10 mg to about 2500 mg twice daily (bid) or three times daily (tid);
(j) a moxifloxacin,
optionally administered to the individual in need thereof at a dose of between about 10 mg to about 2500 mg per day,
(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally administered to the individual in need thereof at a dose of between about 15 mg to about 50 mg per day, or at about 25 to 5000 mg per day;
(l) a furazolidone,
optionally administered to the individual in need thereof at a dose of between about 5 to about 6000 mg/d;
(m) a rifabutin,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,
(n) a nitazoxanide,
optionally administered to the individual in need thereof at a dose of between about 50 to 2000 mg/day or between about 50 to 2000 mg bid;
(o) a furazolidine,
optionally administered to the individual in need thereof at a dose of between about 5 mg to about 6000 mg per day;
(p) a bismuth, bismuth salicylate or bismuth subsalicylate,
optionally administered to the individual in need thereof at a dose of between about 100 mg to about 4000 mg per day;
(q) tetracycline,
optionally administered to the individual in need thereof at a dose of between about 60 mg to 5000 mg per day;
(r) a doxycycline,
optionally administered to the individual in need thereof at a dose of between about 60 mg to 5000 mg per day;
(s) a minocycline, optionally administered to the individual in need thereof at a dose of between about 60 to about 5000 mg daily;
(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or
(u) any combination of (a) to (t) further comprising a clarithromycin, wherein optionally the clarithromycin is administered at a dose of between about 500 mg to 5 gm per day, or 250 mg to 6 gm per day,
wherein optionally the antimicrobial or antibiotic composition is administered together with, before and/or after administration of the potassium competitive acid blocker, the acid pump blocker or the inhibitor of H+/K+ ATPase, and optionally the antimicrobial or antibiotic composition is, formulated together with, or separately from, the potassium competitive acid blocker, the acid pump blocker or the inhibitor of H+/K+ ATPase.

3. The method of claim 1, wherein the drug combination is or comprises:
(1) tegoprazan, revaprazan, linaprazan or soraprazan; and, amoxicillin or amoxicillin/clavulanic acid;
(2) tegoprazan, revaprazan, linaprazan or soraprazan; and, clarithromycin;
(3) tegoprazan, revaprazan, linaprazan or soraprazan; clarithromycin; and, amoxicillin or amoxicillin/clavulanic acid;
(4) tegoprazan, revaprazan, linaprazan or soraprazan; and, rifabutin;
(5) tegoprazan, revaprazan, linaprazan or soraprazan; clarithromycin; and, rifabutin;
(6) tegoprazan, revaprazan, linaprazan or soraprazan; amoxicillin or amoxicillin/clavulanic acid; and, rifabutin;
(7) tegoprazan, revaprazan, linaprazan or soraprazan; amoxicillin or amoxicillin/clavulanic acid; clarithromycin; and, rifabutin;
(8) tegoprazan, revaprazan, linaprazan or soraprazan; and, levofloxacin;
(9) tegoprazan, revaprazan, linaprazan or soraprazan; clarithromycin; and, levofloxacin;
(10) tegoprazan, revaprazan, linaprazan or soraprazan; amoxicillin or amoxicillin/clavulanic acid; and, levofloxacin;
(12) tegoprazan, revaprazan, linaprazan or soraprazan; amoxicillin or amoxicillin/clavulanic acid; clarithromycin; and, levofloxacin;
(13) tegoprazan, revaprazan, linaprazan or soraprazan; amoxicillin or amoxicillin/clavulanic acid; rifabutin; and, levofloxacin;
(14) tegoprazan, revaprazan, linaprazan or soraprazan; amoxicillin or amoxicillin/clavulanic acid; rifabutin; clarithromycin; and, levofloxacin;
(15) tegoprazan, revaprazan, linaprazan or soraprazan; amoxicillin or amoxicillin/clavulanic acid; rifabutin; and, tetracycline;
(16) tegoprazan, revaprazan, linaprazan or soraprazan; amoxicillin or amoxicillin/clavulanic acid; rifabutin; clarithromycin; and, tetracycline;
(17) tegoprazan, revaprazan, linaprazan or soraprazan; and, tetracycline;
(18) tegoprazan, revaprazan, linaprazan or soraprazan; clarithromycin; and, tetracycline;
(19) tegoprazan, revaprazan, linaprazan or soraprazan; and, metronidazole or tinidazole;
(20) tegoprazan, revaprazan, linaprazan or soraprazan; clarithromycin; and, metronidazole or tinidazole;
(21) tegoprazan, revaprazan, linaprazan or soraprazan; and, a bismuth, bismuth salicylate or bismuth subsalicylate;
(22) tegoprazan, revaprazan, linaprazan or soraprazan; clarithromycin; and, a bismuth, bismuth salicylate or bismuth subsalicylate;
(23) tegoprazan, revaprazan, linaprazan or soraprazan; tetracycline; and, a bismuth, bismuth salicylate or bismuth subsalicylate;
(24) tegoprazan, revaprazan, linaprazan or soraprazan; tetracycline; clarithromycin; and, a bismuth, bismuth salicylate or bismuth subsalicylate;
(25) tegoprazan, revaprazan, linaprazan or soraprazan; tetracycline; a bismuth, bismuth salicylate or bismuth subsalicylate; and, metronidazole or tinidazole;
(26) tegoprazan, revaprazan, linaprazan or soraprazan; tetracycline; a bismuth, bismuth salicylate or bismuth subsalicylate; clarithromycin; and, metronidazole or tinidazole;
(27) tegoprazan, revaprazan, linaprazan or soraprazan; tetracycline; and, metronidazole or tinidazole;
(28) tegoprazan, revaprazan, linaprazan or soraprazan; tetracycline; clarithromycin; and, metronidazole or tinidazole;
(29) tegoprazan, revaprazan, linaprazan or soraprazan; and, ampicillin;
(30) tegoprazan, revaprazan, linaprazan or soraprazan; clarithromycin; and, ampicillin;
(31) tegoprazan, revaprazan, linaprazan or soraprazan; tetracycline; metronidazole or tinidazole; and, ampicillin;
(32) tegoprazan, revaprazan, linaprazan or soraprazan; tetracycline; metronidazole or tinidazole; clarithromycin; and, ampicillin;
(33) tegoprazan, revaprazan, linaprazan or soraprazan; tetracycline; and, ampicillin;
(34) tegoprazan, revaprazan, linaprazan or soraprazan; tetracycline; clarithromycin; and, ampicillin;
(35) tegoprazan, revaprazan, linaprazan or soraprazan; and, rifabutin; tegoprazan, revaprazan, linaprazan or soraprazan; clarithromycin; and, rifabutin;
(36) tegoprazan, revaprazan, linaprazan or soraprazan; and, rifampicin;
(37) tegoprazan, revaprazan, linaprazan or soraprazan; clarithromycin; and, rifampicin;
(38) tegoprazan, revaprazan, linaprazan or soraprazan; rifabutin; and, rifampicin;
(39) tegoprazan, revaprazan, linaprazan or soraprazan; rifabutin; clarithromycin; and, rifampicin;
(40) tegoprazan, revaprazan, linaprazan or soraprazan; rifabutin; tetracycline, and, rifampicin;
(41) tegoprazan, revaprazan, linaprazan or soraprazan; rifabutin; tetracycline, clarithromycin; and, rifampicin;
(42) tegoprazan, revaprazan, linaprazan or soraprazan; and, clarithromycin; tegoprazan, revaprazan, and, clarithromycin;
(43) tegoprazan, revaprazan, linaprazan or soraprazan; rifabutin; and, clarithromycin;
(44) tegoprazan, revaprazan, linaprazan or soraprazan; and, clarithromycin;
(45) tegoprazan, revaprazan, linaprazan or soraprazan; rifampicin; and, clarithromycin;
(46) rifampicin; and, clarithromycin;
(47) tegoprazan, revaprazan, linaprazan or soraprazan; and, azithromycin;
(48) tegoprazan, revaprazan, linaprazan or soraprazan; clarithromycin; and, azithromycin;
(49) tegoprazan, revaprazan, linaprazan or soraprazan; rifampicin; and, azithromycin;
(50) tegoprazan, revaprazan, linaprazan or soraprazan; clarithromycin; rifampicin; and, azithromycin;
(51) tegoprazan, revaprazan, linaprazan or soraprazan; rifabutin; and, azithromycin;
(52) tegoprazan, revaprazan, linaprazan or soraprazan; rifabutin; clarithromycin; and, azithromycin;
(53) tegoprazan, revaprazan, linaprazan or soraprazan; rifabutin; and, a vonoprazan or a vonoprazan fumarate;
(54) tegoprazan, revaprazan, linaprazan or soraprazan; rifabutin; clarithromycin; and, a vonoprazan or a vonoprazan fumarate;
(55) tegoprazan, revaprazan, linaprazan or soraprazan; rifabutin; and, metronidazole;
(55) tegoprazan, revaprazan, linaprazan or soraprazan; rifabutin; clarithromycin; and, metronidazole;
(56) tegoprazan, revaprazan, linaprazan or soraprazan; rifabutin; and, tinidazole;
(57) tegoprazan, revaprazan, linaprazan or soraprazan; rifabutin; clarithromycin; and, tinidazole;
(58) tegoprazan, revaprazan, linaprazan or soraprazan; rifabutin; and, nitazoxanide;
(59) tegoprazan, revaprazan, linaprazan or soraprazan; rifabutin; clarithromycin; and, nitazoxanide;
(60) tegoprazan, revaprazan, linaprazan or soraprazan; rifabutin; and, furazolidine;
(61) tegoprazan, revaprazan, linaprazan or soraprazan; rifabutin;
clarithromycin; and, furazolidine;
(62) tegoprazan, revaprazan, linaprazan or soraprazan; and, ciprofloxacin;
(63) tegoprazan, revaprazan, linaprazan or soraprazan; clarithromycin; and, ciprofloxacin;
(64) tegoprazan, revaprazan, linaprazan or soraprazan; rifabutin; and, ciprofloxacin;
(65) tegoprazan, revaprazan, linaprazan or soraprazan; rifabutin; clarithromycin; and, ciprofloxacin
(66) tegoprazan, revaprazan, linaprazan or soraprazan; and, doxycycline or minocycline; or
(67) tegoprazan, revaprazan, linaprazan or soraprazan; clarithromycin; and, doxycycline or minocycline,
wherein optionally the antimicrobial or antibiotic composition is administered together with, before and/or after administration of the potassium competitive acid blocker, the acid pump blocker or the inhibitor of H+/K+ ATPase, and optionally the antimicrobial or antibiotic composition is, formulated together with, or separately from, the potassium competitive acid blocker, the acid pump blocker or the inhibitor of H+/K+ ATPase.

4. The method of claim 1, wherein the drug combination comprises a potassium competitive acid blocker and amoxicillin,
wherein optionally the amoxicillin is administered in an amount of at least 3 g/day,
and optionally the potassium competitive acid blocker is selected from the group consisting of tegoprazan, revaprazan, and linaprazan, and optionally the antimicrobial or antibiotic composition is administered together with, before and/or after administration of the potassium competitive acid blocker, the acid pump blocker or the inhibitor of H+/K+ ATPase,
and optionally the antimicrobial or antibiotic composition is, formulated together with, or separately from, the potassium competitive acid blocker, the acid pump blocker or the inhibitor of H+/K+ ATPase.

5. (canceled)

6. The method of claim 1, wherein the drug combination comprises a potassium competitive acid blocker, amoxicillin, and clarithromycin, and optionally the potassium competitive acid blocker is selected from the group consisting of tegoprazan, revaprazan, and linaprazan.

7. The method of claim 1, wherein the drug combination comprises a potassium competitive acid blocker, amoxicillin, and a rifamycin derivative, and optionally the potassium competitive acid blocker is selected from the group consisting of tegoprazan, revaprazan, and linaprazan.

8. The method of claim 7, wherein the rifamycin derivative is rifabutin.

9. (canceled)

10. The method of claim 1, wherein the drug combination is:
(i) tegoprazan, amoxicillin, and clarithromycin;
(ii) tegoprazan and amoxicillin; or
(iii) revaprazan, amoxicillin, and rifabutin.

11. The method of claim 1, wherein the drug combination comprises a potassium competitive acid blocker, a tetracycline antibiotic, a nitroimidazole antibiotic, and a bismuth salt.

12. The method of claim 11, wherein the drug combination comprises linaprazan, tetracycline, tinidazole, and bismuth subsalicylate.

13. The method of claim 1, wherein the drug combination is administered to the individual in need thereof for about 2, 3, 4, 5, 6, 7, 8, 9, 10, 11, 12, 13 or 14 or more days.

14-15. (canceled)

16. The method of claim 1, wherein the drug combination is contained in or comprises one or more of a formulation, a pharmaceutical preparation or a pharmaceutical composition.

17. The method of claim 1, wherein any one or several of the potassium competitive acid blocker or the acid pump blocker, or the inhibitor of H+/K+ ATPase, or the antimicrobial or antibiotic composition is administered to the individual in need thereof:
(a) at a unit dosage of between about 5 mg to about 5000 mg per day, or
(b) in a unit dosage form of between about 10 mg and 200 mg, or between about between about 40 mg and 100 mg, or between about between about 100 mg and 500 mg, or between about between about 500 mg and 1000 mg, or is about 10, 20, 30, 40, 50, 60, 70, 75, 80, 90 or 100 mg per unit dose,
which optionally can be administered once a day, bid or tid, or a four times a day, five times a day or six times a day or more, regimen.

18. The method of claim 1, wherein the drug combination:
(a) is formulated as a chewable delivery vehicle, a gum, a gummy, a candy, a lozenge, an ice cream or an ice, or a yogurt;
(b) further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof;
(c) further comprises a preservative, a benzoic acid or a potassium sorbate;
(d) further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents;
(d) further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch;
(e) further comprises an additive selected from one or more of a saline, a media, a defaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient;
(f) further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin atorvastatin, pravastatin, fluvastatin or rosuvastatin, cranberry components, curcumin, silver nanoparticles, Acetyl-11-keto-β-boswellic acid (AKBA), barley coffee components, probiotics, sinefunqin, 5-adenosylmethionine, 5-adenosyl-homocysteine, Delisea furanones, N-sulfonyl homoserine lactones or any combination thereof;
(g) is formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation;
(h) is contained in a delivery vehicle, product of manufacture, container, syringe, device or bag; or
(i) is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

19-27. (canceled)

28. A drug combination comprising:
(a) the drug combination as defined in the method of any one of the preceding claims; or
(b) [symbol]
(i) a drug combination comprising at least one potassium-competitive acid blocker (PCAB) or acid pump blocker (or acid pump antagonist (APA)), or at least one inhibitor of H+/K+ ATPase,
wherein optionally the at least one potassium competitive acid blocker is or comprises:
tegoprazan, or 7-[[(45)-5,7-difluoro-3,4-dihydro-2H-chromen-4-yl]oxy]-N,N,2-trimethyl-3H-benzimidazole-5-carboxamide, or equivalent,
revaprazan or N-(4-fluorophenyl)-4,5-dimethyl-6-(I-methyl-I,2,3,4-tetrahydroisoquinolin-2-yl)pyrimidin-2-amine hydrochloride or equivalent,
linaprazan, or 8-[(2,6-dimethylphenyl)methylamino]-N-(2-hydroxyethyl)-2,3-dimethylimidazo[I,2-a]pyridine-6-carboxamide, or equivalent,
a compound having the structure of Formula 1, or equivalent, 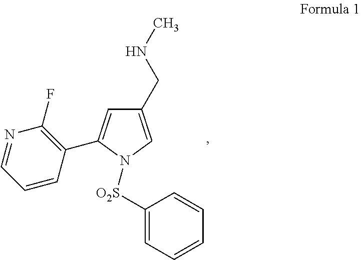 [SMILES: CNCc1cc(-c2cccnc2F)n(S(=O)(=O)c2ccccc2)c1]
a compound having the structure of Formula 2, or equivalent, 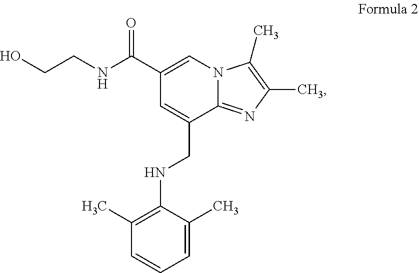 [SMILES: Cc1cccc(C)c1NCc1cc(C(=O)NCCO)cn2c(C)c(C)nc12]
and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[I,2-h][I,7]naphthyridin-8-ol, or equivalent; and
(ii) [symbol]
(a) at least one penicillin or a penicillin derivative or beta-lactamase inhibitor, wherein optionally the at least one penicillin derivative or beta-lactamase inhibitor comprises: an amoxicillin; a clavulanate, potassium clavulanate, or clavulanic acid; an ampicillin; a sulbactam; a tazobactam; a ticarcillin; a piperacillin; or equivalents thereof,
and optionally the penicillin, penicillin derivative or beta-lactamase inhibitor is administered to the individual in need thereof at a dose of from between about 100 mg to 250 mg, to about 3 grams (g), twice daily (bid), or at a dose of from between about 100 mg to 250 mg, to about 3 grams (g) three times daily (tid),
and optionally the amoxicillin is formulated as an amoxicillin/clavulanic acid combination,
and optionally the potassium clavulanate or clavulanic acid is formulated with ticarcillin,
and optionally the ampicillin is formulated with sulbactam;
and optionally the piperacillin is formulated with tazobactam;
(b) a rifampicin or rifampin,
optionally administered to the individual in need thereof at a dose of between about 10 mg to about 450 mg bid or tid;
(c) a clarithromycin,
optionally administered to the individual in need thereof at a dose of between about 10 mg to about 3 g twice daily (bid) or tid, or at a dose of between about 500 mg to 5 gm per day,
(d) an azithromycin,
optionally administered to the individual in need thereof at a dose of between about 100 mg to 3 grams bid, or once a day, or every second or third day;
(e) a vonoprazan or a vonoprazan fumarate, or a 5-(2-fluorophenyl)-1-(pyridin-3-ylsulfonyl)-IH-pyrrol-3-yl)-N-methylmethanamine monofumarate, or a 1-(5-(2-fluorophenyl)-I-(pyridin-3-ylsulfonyl)-IH-pyrrol-3-yl)-N-methyl-methanamine fumarate),
optionally administered to the individual in need thereof at a dose range of between about 1 mg to about 100 mg twice daily (bid), or for about 10 mg to 25 mg or more twice daily (bid);
(f) a metronidazole,
optionally administered to the individual in need thereof at a dose of between about 10 mg to 20 mg, to about 1000 mg, three times daily (tid), or at a dose range of between about 10 to 2000 mg or more three times daily (tid);
(g) a tinidazole,
optionally administered to the individual in need thereof at a daily dose of between about 50 mg to 3 grams;
(h) a levofloxacin,
optionally administered to the individual in need thereof at a dose of between about 10 mg to about 5000 mg twice daily (bid) or three times daily (tid);
(i) a ciprofloxacin,
optionally administered to the individual in need thereof at a dose of between about 10 mg to about 2500 mg twice daily (bid) or three times daily (tid);
(j) a moxifloxacin,
optionally administered to the individual in need thereof at a dose of between about 10 mg to about 2500 mg per day,
(k) a TG44, or a 1-1000 mg/d] {[4-methylbenzyl 4′-[trans-4-(guanidine-methyl) cyclohexyl carbonyloxy] biphenyl-4-carboxylate monohydrochloride}, or CAS registry number 178748-55-5,
optionally administered to the individual in need thereof at a dose of between about 15 mg to about 50 mg per day, or at about 25 to 5000 mg per day;
(l) a furazolidone,
optionally administered to the individual in need thereof at a dose of between about 5 to about 6000 mg/d;
(m) a rifabutin,
optionally administered to the individual in need thereof at a dose of between about 10 to about 4500 mg/d,
and optionally the rifabutin dose is ramped up starting at about 40 to about 60 g bid or tid, and optionally rising over 3 days to about 200 to about 450/d,
(n) a nitazoxanide,
optionally administered to the individual in need thereof at a dose of between about 50 to 2000 mg/day or between about 50 to 2000 mg bid;
(o) a furazolidine,
optionally administered to the individual in need thereof at a dose of between about 5 mg to about 6000 mg per day;
(p) a bismuth, bismuth salicylate or bismuth subsalicylate,
optionally administered to the individual in need thereof at a dose of between about 100 mg to about 4000 mg per day;
(q) tetracycline,
optionally administered to the individual in need thereof at a dose of between about 60 mg to 5000 mg per day;
(r) a doxycycline,
optionally administered to the individual in need thereof at a dose of between about 60 mg to 5000 mg per day;
(s) a minocycline,
optionally administered to the individual in need thereof at a dose of between about 60 to about 5000 mg daily;
(t) any combination of (a) to (s), wherein optionally the therapeutic combination comprises any one, two, three or four of more of (a) to (u) in combination with a potassium competitive acid blocker or an acid pump blocker, or an inhibitor of H+/K+ ATPase; or
(u) any combination of (a) to (t) further comprising a clarithromycin, wherein optionally the clarithromycin is administered at a dose of between about 500 mg to 5 gm per day, or 250 mg to 6 gm per day.

29. A kit or product of manufacture comprising a drug combination as set forth in in claim 1.

30-31. (canceled)

32. The method of claim 1, wherein the at least one potassium competitive acid blocker is or comprises:
(a) tegoprazan, or 7-[[(4S)-5,7-difluoro-3,4-dihydro-2H-chromen-4-yl]oxy]-N,N,2-trimethyl-3H-benzimidazole-5-carboxamide, or equivalent,
(b) revaprazan (optionally REVANEX™) or N-(4-fluorophenyl)-4,5-dimethyl-6-(I-methyl-I,2,3,4-tetrahydroisoquinolin-2-yl)pyrimidin-2-amine hydrochloride or equivalent,
(c) linaprazan, or 8-[(2,6-dimethylphenyl)methylamino]-N-(2-hydroxyethyl)-2,3-dimethylimidazo[I,2-a]pyridine-6-carboxamide, or equivalent,
(d) a compound having the structure of Formula 1, or equivalent, 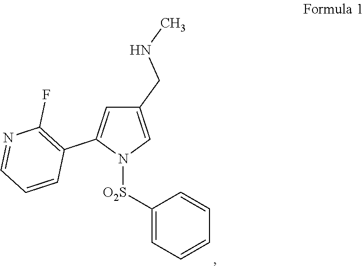 [SMILES: CNCc1cc(-c2cccnc2F)n(S(=O)(=O)c2ccccc2)c1 ; ","]
(e) a compound having the structure of Formula 2, or equivalent, 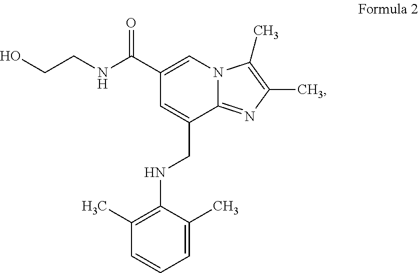 [SMILES: Cc1cccc(C)c1NCc1cc(C(=O)NCCO)cn2c(C)c(C)nc12]
and optionally the at least one inhibitor of H+/K+ ATPase is soraprazan, or (7R,8R,9R)-7-(2-methoxyethoxy)-2,3-dimethyl-9-phenyl-7,8,9,10-tetrahydroimidazo[I,2-h][I,7]naphthyridin-8-ol, or equivalent, or
(f) any combination of (a) to (e).
